(19) United States
(12) Patent Application Publication
(10) Pub. No.: US 2024/0123437 A1
(43) Pub. Date: Apr. 18, 2024

(54) CHEMICALLY STABLE ION CONDUCTORS AND USES THEREOF

(71) Applicant: Technion Research & Development Foundation Limited, Haifa (IL)

(72) Inventors: Charles Eliezer DIESENDRUCK, Haifa (IL); Dario R. DEKEL, Haifa (IL)

(73) Assignee: Technion Research & Development Foundation Limited, Haifa (IL)

(21) Appl. No.: 18/269,974

(22) PCT Filed: Dec. 31, 2021
(86) PCT No.: PCT/IL2021/051562

§ 371 Date: Jun. 28, 2023

Related U.S. Application Data
(60) Provisional application No. 63/132,561, filed on Dec. 31, 2020.

(51) Int. Cl. B01J 41/14; B01J 41/05; B01J 47/12; C07D 209/44; C08F 212/08; C08J 5/22; H01M 8/083; H01M 8/103

(52) U.S. Cl. CPC B01J 41/14; B01J 41/05; B01J 47/12; C07D 209/44; C08F 212/08; C08J 5/2231; C08J 5/2243; H01M 8/083; H01M 8/103; C08F 2800/10; C08J 2323/06; C08J 2325/08; H01M 2008/1095

(57) ABSTRACT

Polymeric ion conductors composed of a polymeric matrix associated with an isoindolinium moiety represented by Formula I as described in the specification are provided. Anion conducting compositions comprising an anion conducting polymer associated with, or threaded within and having mechanically interlocked therearound, a cyclic moiety are also provided. lonomeric materials, including, for example, ion exchange membranes such as anion exchange membranes, made of the polymeric ion conductor and/or the anion conducting composition, and electrochemical systems and articles-of-manufacturing containing the polymeric ion conductor, the anion conducting composition or the ion exchange membrane are also provided.

### RELATED APPLICATION/S

This application claims the benefit of priority under 35 USC §119(e) of U.S. Provisional Patent Application No. 63/132,561 filed Dec. 31, 2020, the contents of which are incorporated herein by reference in their entirety.

### GOVERNMENT LICENSE RIGHTS

This invention was made with government support under contract W911NF1810322 awarded by the Department of the Army. The government has certain rights in the invention.

### FIELD AND BACKGROUND OF THE INVENTION

The present invention, in some embodiments thereof, relates generally to energy conversion and, more particularly, but not exclusively, to novel methodologies for improving the chemical stability of ion conductors such as, but not limited to, ion conductions used for forming ion exchange membranes and electrochemical systems and other articles of manufacturing containing same. Some embodiments of the present invention relate to a novel family of quaternary ammonium compounds and to their use as ionic conductors for forming ion exchange membranes and electrochemical systems and other articles of manufacturing containing same. Other embodiments of the present invention relate to a use of cyclic moieties associated with ion conducting polymers (e.g., cyclic moieties mechanically interlocked with positively-charged polymers such as polyrotaxanes) for forming ion exchange membranes and electrochemical systems and other articles of manufacturing containing same.

Over the recent decades, renewable energy sources have slowly become more present given their lower environmental impact. Considering factors such as transportation and energy density, one of the most suitable ways to store this energy is by converting it into chemical feedstocks such as hydrogen and methanol.

Fuel cells (FCs) can use these feedstocks for generation of electricity when needed. FCs have been considered as one of the most efficient and pollution free power generation technology, being not only “pollution-free” but also capable of manifesting more than twice the efficiency of traditional combustion technologies.

Fuel cells (FCs) gained increasing importance during the last decades, mostly as a potential technology for transport applications. Among the numerous advantages, safe and reliable operation, environmental friendliness, high efficiency and power density as well as rapid refueling have marked fuel cells as one of the most promising green energy conversion technologies.

Ion exchange membranes are a key component of and in numerous energy storage and conversion devices, including fuel cells. They play important roles, including separation between anode and cathode, ion transportation, fuel separation and deterrence of impurities.

Several inorganic (e.g. ceramics) as well as organic polymeric membranes have been developed and studied, and each present unique advantages and limitations. Currently, proton exchange membrane fuel cells (PEMFCs) are an efficient method for providing power in various applications, ranging from automotive transportation to portable power generation. However, the extensive commercialization of PEMFCs remains a challenge due to their high cost and limited durability.

Alkaline fuel cells offer many advantages over acidic fuel cells, the main advantage being the possibility of using different metals (as opposed to platina) as electrodes.

Anion exchange membrane fuel cells (AEMFCs) are an expedient alternative to PEMFCs and are currently gathering modernized attention. AEMFCs are currently hindered by the challenging requirements of its anion exchange membrane (AEM): good thermal and mechanical stability, high hydroxide ion conductivity, and long-term durability at elevated temperatures in strong alkaline conditions.

Anion exchange membrane fuel cells (AEMFCs) use cation-functionalized hydroxide conductive membranes. AEMFCs have been gaining increased attention due to their operation in alkaline media which eliminates the requirements for expensive platinum catalysts. Yet, despite the numerous advantages of AEMFCs, the short lifetime of the polymeric electrolyte is a major deterrent for the practical application of this technology. The high basicity and nucleophilicity of the hydroxide anion (OH⁻), especially in the operating temperatures of these cells (typically 60° C.-80° C.), leads to rapid degradation of the cationic groups that provide the anion transportation ability of the polymer electrolyte, resulting in voltage losses and subsequent power density drop.

Currently, the best performing membranes can live only for a few tens of hours.

### Quaternary Ammonium-Based ion Conductors

Currently practiced anion exchange-membranes (AEMs) typically use quaternary ammonium salts (QAs) for ion conductance.

Quaternary ammonium (QAs) salts have been the most explored cations for this application, given their relatively high stability to alkaline conditions compared to the more oxophilic phosphonium and sulfonium salts [Luo et al. J. Memb. Sci. 2018, 555, 429-454; Gu et al. Angew. Chem. Int. Ed. 2009, 48, 6499-6502; Noonan et al. J. Am. Chem. Soc. 2012, 134, 18161-18164; Zhang et al. RSC Adv. 2012, 2, 12683-12685].

However, in the presence of hydroxide, these groups are prone to undergo Hofmann elimination or nucleophilic attack (S_{N}2) on the α-carbon.

That said, the fact that the OH⁻is the conducting anion and at the same time responsible for the degradation of the functionalized polymer, seems to be an unsolvable challenge so far, as no membrane currently can sustain the required operating conditions for sufficient time, and at the desired power.

Many efforts have been put to develop better quaternary ammonium salts (QAs) in order to make longer lived membranes, but, while there have been numerous improvements in power, the lifetime is still in the same order of magnitude.

Current ammonium salts perform very well in aqueous alkaline media, even at high temperatures, but fail badly in a relatively dry alkaline environment. The leading QA in dry environment is benzyltrimethyl ammonium salt (BTMA). See, for example, Dekel et al., Chem. Mater. 2017, 29 (10), 4425-4431.

There are numerous strategies that have been applied to improve the alkaline stability of Anion Exchange Membranes (AEMs) by either altering the nature of the quaternary ammonium ion (QA) or manipulating the architecture of the polymer backbone [Dekel, D. R. J. Power Sources 2018, 375, 158-169].

Recent studies have suggested that these mechanisms could be completely eliminated by creating a QA with only sp² carbons connected to the nitrogen atom. However, it has been shown that also in such cases the hydroxide ions still degrade the QA, by acting as a reducing agent, through an inner sphere single electron transfer followed by a radical coupling [Aharonovich et al. Synlett 2018, 29 (10), 1314-1318; Gjineci et al. European J. Org. Chem. 2020, 2020 (21), 3161-3168]. Improving the performance of such QAs by altering the electronic properties using substituent effects has also been suggested, and was shown to lead to a substantial increase in the alkaline stability of this type of QAs. Reference is also made to WO 2020/044348.

Substituent effects have been shown to be a key tool in improving the stability of other QAs such as imidazolium salts [see, for example, Hugar et al. J. Am. Chem. Soc. 2015, 137 (27), 8730-8737; Lin et al. Chem. Mater. 2013, 25 (9), 1858-1867; Fan et al. Nat. Commun. 2019, 10 (1), 2306].

### Mechanically Interlocked Polymers

Mechanically interlocked polymers, such as polyrotaxanes and polycatenanes, have been a focus of polymer chemists in recent years, given their fascinating properties as a consequence of the dynamic nature of the mechanical bond. The ability of the subcomponents to shuttle or rotate relative to each other have enabled their use as molecular switches and molecular machines.

In addition, these polymers have shown a few advantages in materials for energy applications. Polyrotaxanes using cyclodextrins have exhibited substantial ionic conductivity and superior oxidative electrochemical stability when used as solid-electrolytes in lithium-ion batteries [Imholt et al. ACS Macro Lett. 2018, 7(7), 881-885; Imholt et al. J. Power Sources 2019, 409, 148-158], and demonstrated higher elasticity and lower volume expansion when used as binders in Si anodes [Choi et al. Science 2017, 357 (6348), 279-283].

Rotaxanes using crown ethers have been used as proton exchange membranes, showing proton conductivity higher than Nafion's [Ge et al. Nat. Commun. 2018, 9, 2297].

Crown ether based rotaxanes have been used in AEMs in order to affect (increase) the ion conductivity and water uptake compared to pristine polymers without rotaxanes [Ge et al. Adv. Mater. 2016, 28 (18), 3467-3472; Yang et al. ACS Appl. Mater. Interfaces 2020, 12 (22), 24806-24816].

Additional background art includes WO 2009/083509.

### SUMMARY OF THE INVENTION

According to an aspect of some embodiments of the present invention there is provided an ion conductor comprising a polymeric matrix and a quaternary ammonium salt-containing moiety being is association with the matrix, the quaternary ammonium salt-containing moiety being represented by Formula I:

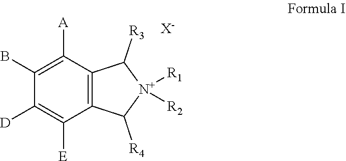

wherein:
R₁ and R₂ are each independently selected from alkyl, cycloalkyl and aryl, or represents an attachment point to the polymeric matrix;
X⁻is a counter anion;
R₃, R₄, A and E are each independently selected from hydrogen, alkyl, halo, haloalkyl, cycloalkyl, aryl, heteroaryl, hydroxy, alkoxy, aryloxy, and any other substituent, or represents an attachment point to the polymeric matrix, provided that at least one of A and E is a bulky substituent capable of imparting steric hindrance around the positively charged portion of the compound; and
B and D are each independently hydrogen, halo, alkyl, haloalkyl, cycloalkyl, aryl, heteroaryl, hydroxy, alkoxy, aryloxy, and any other substituent, or represents an attachment point to the polymeric matrix, provided that at least one of B and D is a bulky substituent that positions one or both of A and E at a configuration that imparts the steric hindrance.

According to some of any of the embodiments described herein, at least one of B and D is other than hydrogen or fluoro.

According to some of any of the embodiments described herein, at least one of B and D is chloro, bromo, or iodo.

According to some of any of the embodiments described herein, at least one of B and D is alkoxy.

According to some of any of the embodiments described herein, at least one of A and E is alkoxy.

According to some of any of the embodiments described herein, at least one of B and D is chloro, bromo, or iodo.

According to some of any of the embodiments described herein, each of A and E is independently an alkoxy.

According to some of any of the embodiments described herein, at least one or both of A and E is independently an aryl.

According to some of any of the embodiments described herein, the ion conductor comprises a polymeric or co-polymeric backbone which comprises a plurality of backbone units, wherein at least a portion of the backbone units comprise the quaternary ammonium salt-containing moiety represented by Formula I, and wherein an attachment point of the moiety to the backbone units in via one or more of A, B, D, E, R₁, R₂, R₃ or R₄.

According to some of any of the embodiments described herein, the quaternary ammonium salt-containing moiety represented by Formula I is covalently attached as a pendant group to the portion of backbone units.

According to some of any of the embodiments described herein, the quaternary ammonium salt-containing moiety represented by Formula I forms a part of the polymeric backbone.

According to some of any of the embodiments described herein, the polymeric or co-polymeric backbone comprises backbone units of one or more of polystyrene, polyethylene (PE), PTFE, polypropylene, polyethylene imide (PEI), polyimide (PI), poly(ethylenetetrafluoroethylene) (ETFE), polyether, epoxy polymer, poly(ethylene oxide) (PEO), poly(phenylene oxide) (PPO), polysulfone, poly(benzimidazole) (PBI) and poly(phenylene) (PP).

According to an aspect of some embodiments of the present invention there is provided an anion conducting composition comprising an ion conductor as described herein in any of the respective embodiments and any combination thereof and a cyclic moiety associated with the ion conductor.

According to some of any of the embodiments described herein, the cyclic moiety is mechanically interlocked around at least a portion of the ion conductor.

According to some of any of the embodiments described herein, the polymeric matrix comprises at least one end-capping moiety at a backbone terminus thereof and/or at a pendant group terminus thereof, and being threaded within a cyclic moiety, wherein the end-capping moiety has a volume larger than a volume of the cyclic moiety to thereby have the cyclic moiety mechanically interlocked around at least a portion of the ion conductor.

According to some of any of the embodiments described herein, the cyclic moiety is a heterocyclic moiety.

According to some of any of the embodiments described herein, the heterocyclic moiety comprises at least one electronegative heteroatom.

According to some of any of the embodiments described herein, the cyclic moiety comprises at least 12, or at least 16, or at least 18, carbon atoms.

According to some of any of the embodiments described herein, the cyclic moiety is a crown ether.

According to an aspect of some embodiments of the present invention there is provided an anion exchange membrane comprising the ion conductor as described herein in any of the respective embodiments and any combination thereof or the anion conducting composition as described herein in any of the respective embodiments and any combination thereof.

According to an aspect of some embodiments of the present invention there is provided an electrochemical system comprising the ion conductor as described herein in any of the respective embodiments and any combination thereof, or the anion conducting composition as described herein in any of the respective embodiments and any combination thereof or the anion exchange membrane as described herein in any of the respective embodiments and any combination thereof.

According to some of any of the embodiments described herein, the electrochemical is a fuel cell.

According to an aspect of some embodiments of the present invention there is provided an article-of-manufacturing comprising the ion conductor as described herein in any of the respective embodiments and any combination thereof or the anion conducting composition as described herein in any of the respective embodiments and any combination thereof or the electrochemical system as described herein in any of the respective embodiments and any combination thereof.

According to an aspect of some embodiments of the present invention there is provided an anion conducting composition comprising an anion conducting polymer featuring at least one end-capping moiety at a backbone terminus thereof and/or at a pendant group terminus thereof and a cyclic moiety, the polymer being threaded within a cyclic moiety, wherein the at least one end-capping moiety has a volume larger than a volume of the cyclic moiety to thereby have the cyclic moiety mechanically interlocked around the linear polymer.

According to some of any of the embodiments described herein, the cyclic moiety is a heterocyclic moiety.

According to some of any of the embodiments described herein, the heterocyclic moiety comprises at least one electronegative heteroatom.

According to some of any of the embodiments described herein, the cyclic moiety comprises at least 12, or at least 16, or at least 18, carbon atoms.

According to some of any of the embodiments described herein, the cyclic moiety is a crown ether.

According to some of any of the embodiments described herein, the anion conducting polymer is selected from a quaternary ammonium salt-containing polymer, a metallopolymer, a sulfonium-containing polymer and a phosphonium-containing polymer.

According to some of any of the embodiments described herein, the cyclic moiety is covalently attached to the polymer.

According to some of any of the embodiments described herein, a stability of the composition when subjected to an alkaline environment is higher than a stability of the anion conducting polymer.

According to some of any of the embodiments described herein, the cyclic moiety is for increasing a stability of the anion conducting polymer when subjected to alkaline environment.

According to an aspect of some embodiments of the present invention there is provided an anion exchange membrane comprising the anion conducting composition as described herein in any of the respective embodiments and any combination thereof.

According to an aspect of some embodiments of the present invention there is provided an electrochemical system comprising the anion conducting composition as described herein in any of the respective embodiments and any combination thereof or the anion exchange membrane as described herein in any of the respective embodiments and any combination thereof.

According to some of any of the embodiments described herein, the electrochemical system is a fuel cell.

According to an aspect of some embodiments of the present invention there is provided an article-of-manufacturing comprising the anion conducting composition of as described herein in any of the respective embodiments and any combination thereof or the electrochemical system as described herein in any of the respective embodiments and any combination thereof.

According to some of any of the embodiments described herein, the article-of-manufacturing is a fuel cell, a battery, an electrolyzer, a disinfectant, an antibacterial product, a cloth, a food package, a packaging of pharmaceutical or cosmetic product, and an anticoagulant-containing and/or antioxidant-containing pharmaceutical product.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

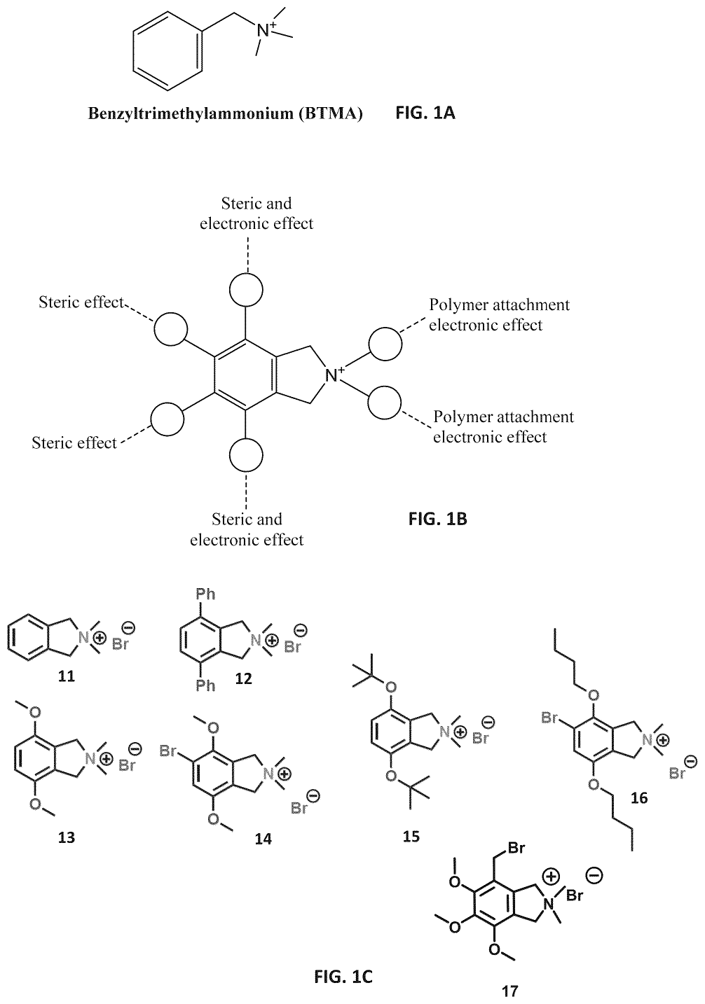

FIGS. 1A-C present the chemical structure of MTBA, a quaternary ammonium salt currently practiced in anion exchange membranes (FIG. 1A), a general structure design of novel, isoindolinium-based quaternary ammonium salts according to some of the present embodiments (FIG. 1B) and chemical structures of exemplary isoindolinium salts according to some embodiments of the present invention (FIG. 1C).

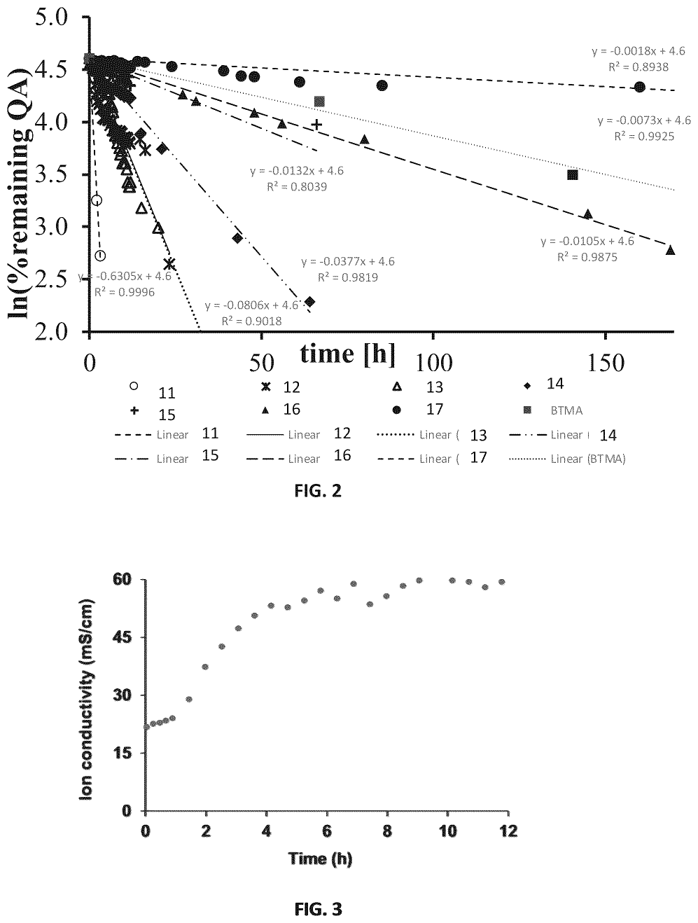

FIG. 2 presents logarithmic plots showing the decomposition kinetics of different exemplary isoindolinium salts under alkaline conditions compared to unsubstituted isoindolinium salt and benzyltrimethyl ammonium salt (BTMA), with a linear fit (pseudo-first order kinetics).

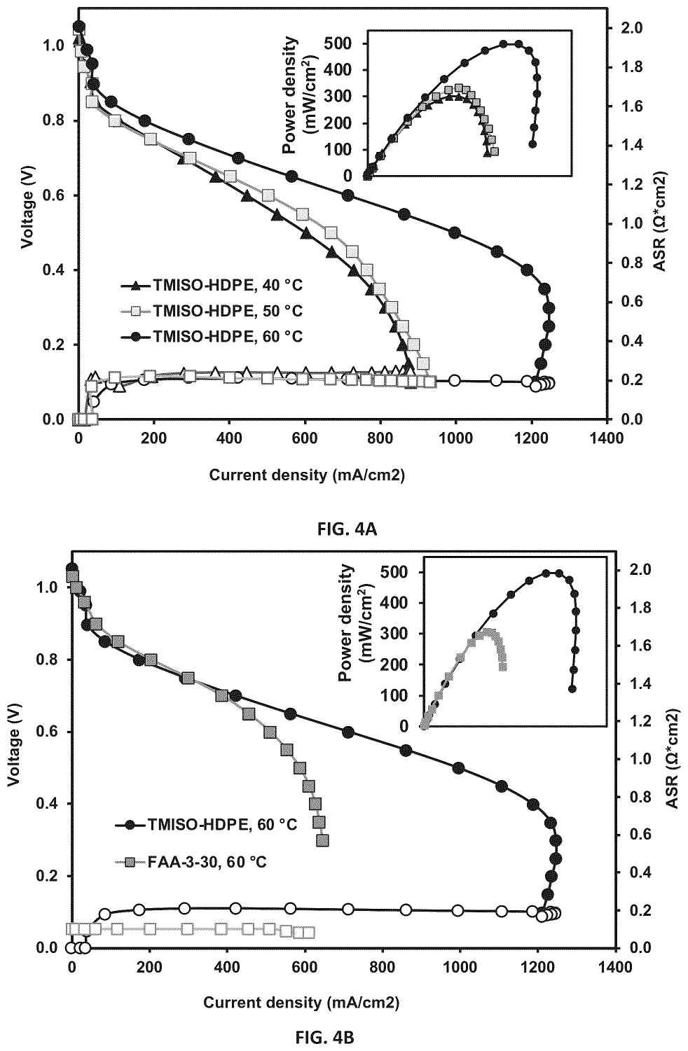

FIG. 3 presents a scattered plot showing ion conductivity of an exemplary isoindolinium-based AEM according to some of the present embodiments, TMISO-HDPE, as a function of time.

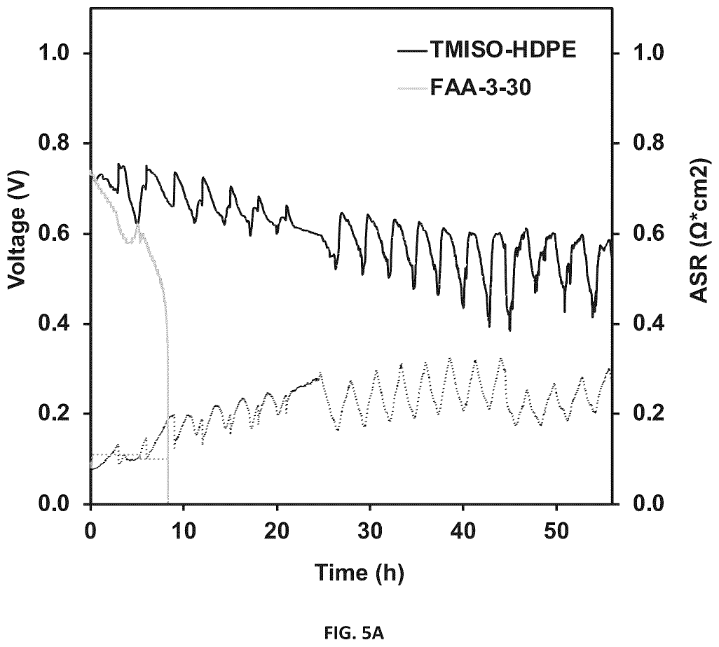

FIGS. 4A-B present comparative plots of long term stability tests showing voltage (filled shapes), area-specific resistance (empty shapes), and power density (insert) as a function of current density. FIG. 4A presents data obtained for an exemplary AEMFC according to some of the present embodiments, TMISO-HDPE, at 40° C. (triangle), 50° C. (square) and 60° C. (dot). FIG. 4B shows a comparison of the data obtained with TMISO-HDPE (circle) and FAA-3-30 (square) at 60° C.

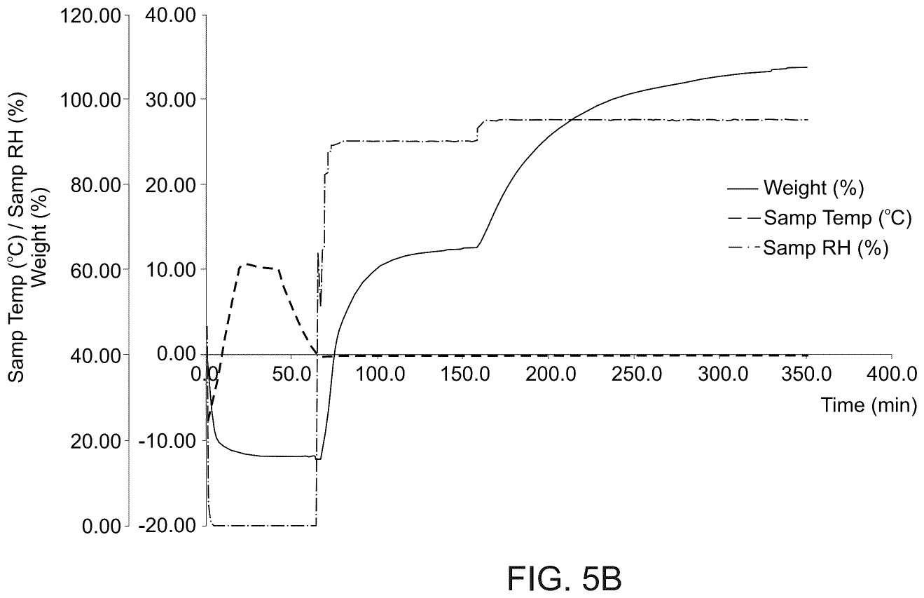

FIGS. 5A-B present graphs showing fuel cell tests of an exemplary AEM according to the present embodiments, TMISO-HDPE. FIGS. 5A presents voltage and area-specific resistance (ASR) as a function of time, which represent a long-term stability test, for AEMFCs operated at 60° C. under a constant current density of 300 mA/cm²; and FIG. 5B presents water uptake plots of TMISO-HDPE showing weight, sample temperature and % RH as a function of time (in minutes).

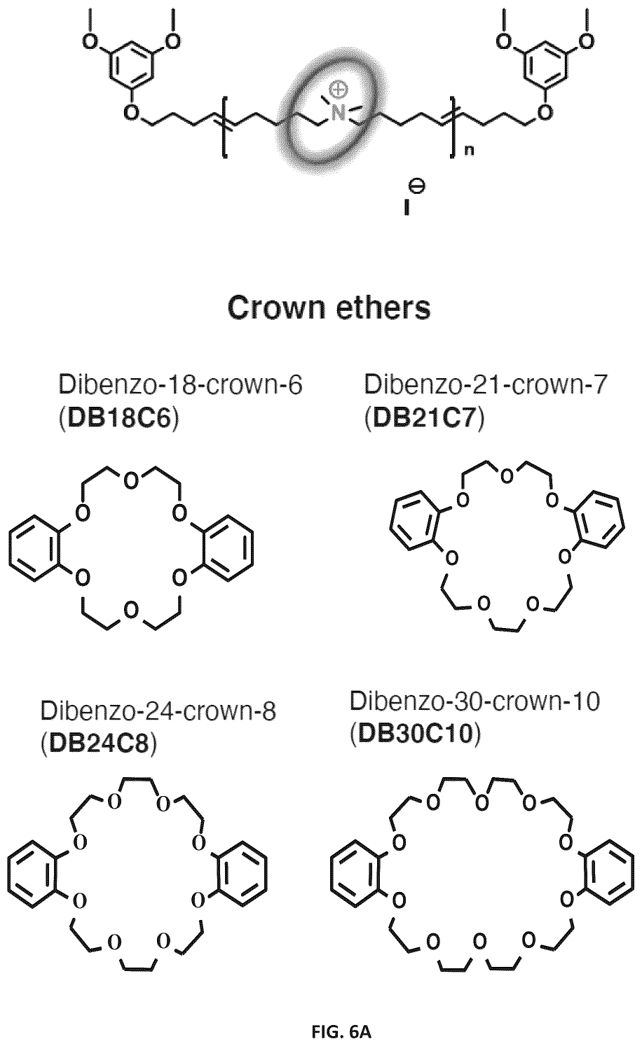

FIGS. 6A-B present the chemical structures of exemplary components of a polyrotaxane according to some embodiments of the present invention; an exemplary end-capped positively-charged polymer and exemplary crown ethers as non-covalent steric shields (FIG. 6A), and an exemplary synthetic protocol for preparing a control positively-charged polymer (8a) and the exemplary polyrotaxanes (8b-8e) (FIG. 6B).

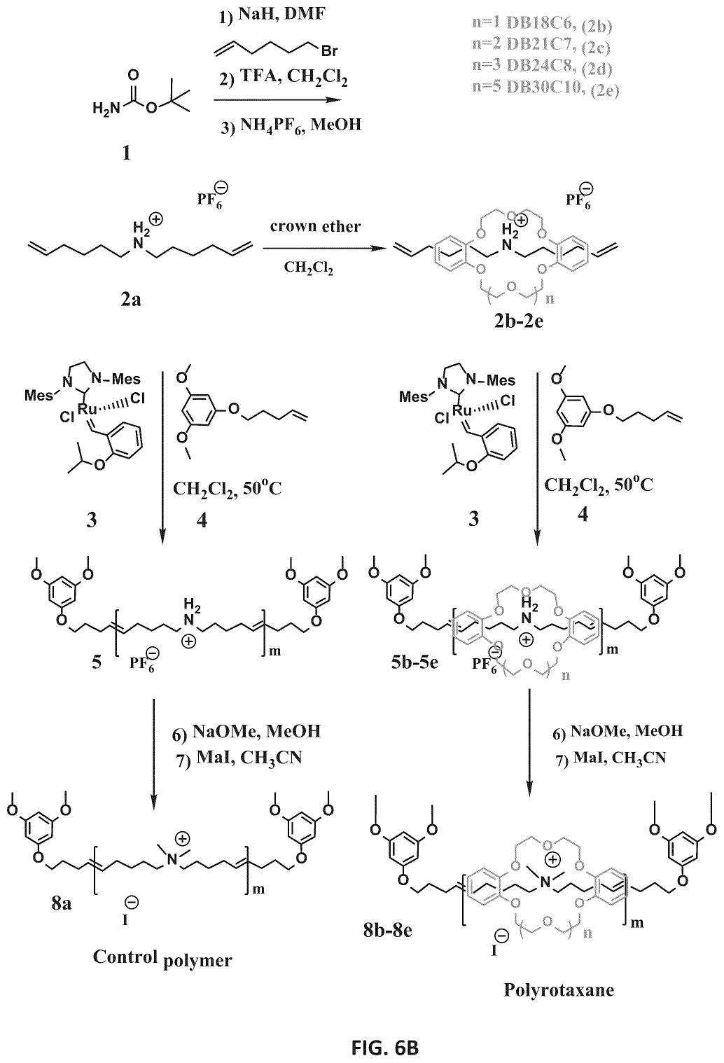

FIGS. 7A-B present comparative plots showing percentage of the remaining control polymer and polyrotaxanes (8a-8e) as a function of time during reaction with 0.5 M OH⁻(λ=4) in DMSO-d₆ at room temperature, as determined by NMR data fitted with exponential (FIG. 7A) and linear (FIG. 7B) trend lines.

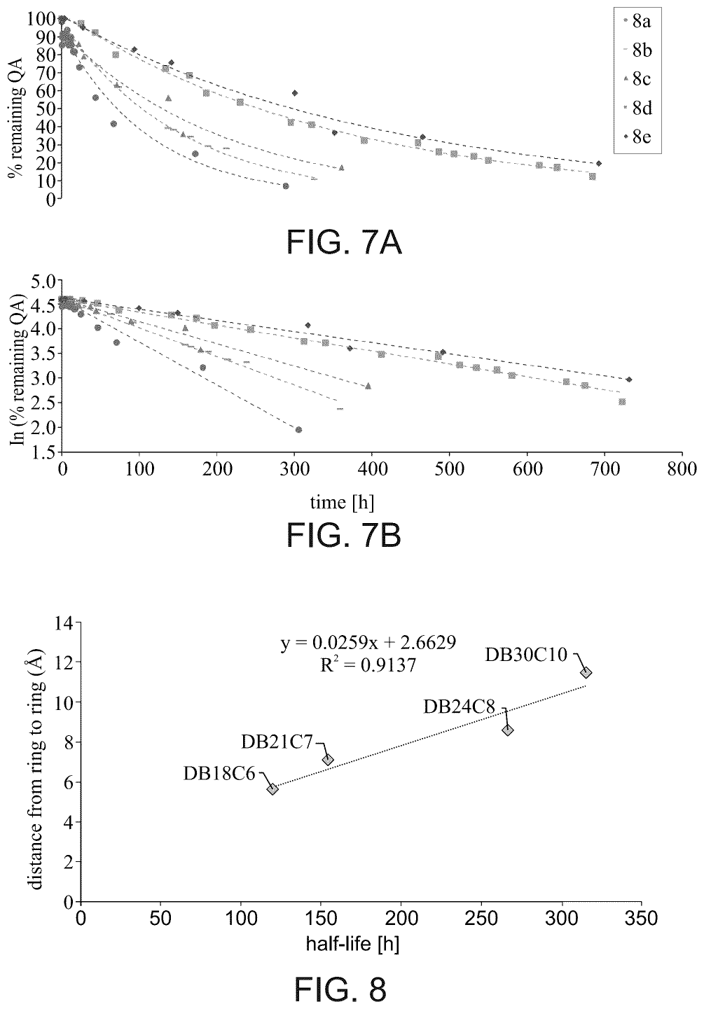

FIG. 8 is a plot showing the correlation of the O—O distance of the crown ether cavities with the half-life of the corresponding polyrotaxane.

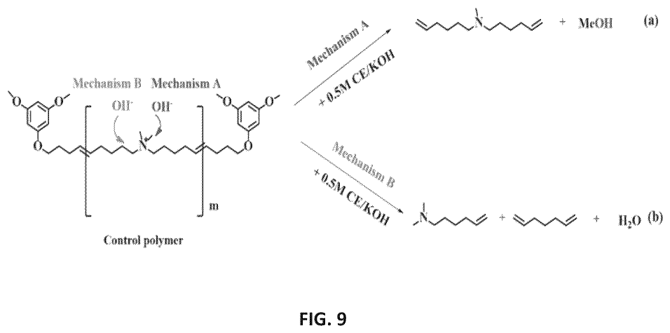

FIG. 9 presents non-limiting proposed degradation schemes for the control unthreaded polymer 8a.

### DESCRIPTION OF SPECIFIC EMBODIMENTS OF THE INVENTION

The present invention, in some embodiments thereof, relates generally to energy conversion and, more particularly, but not exclusively, to novel methodologies for improving the chemical stability of ion conductors such as, but not limited to, ion conductions used for forming ion exchange membranes and electrochemical systems and other articles of manufacturing containing same. Some embodiments of the present invention relate to a novel family of quaternary ammonium compounds and to their use as ionic conductors for forming ion exchange membranes and electrochemical systems and other articles of manufacturing containing same. Other embodiments of the present invention relate to a use of cyclic moieties associated with ion conducting polymers (e.g., cyclic moieties mechanically interlocked with positively-charged polymers such as polyrotaxanes) for forming ion exchange membranes and electrochemical systems and other articles of manufacturing containing same.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Quaternary ammonium salts (QAs) present multiple industrial applications such as surfactants, antibacterial agents and as functional groups in anion exchange membranes (AEM). For the latter application, alkaline stability is key, especially in applications where hydroxide is the anion being transported. In recent years, many advances were done to improve QAs using different substituents, but in applications such as anion exchange membrane fuel cells (AEMFCs), the QA lifetime is still far from what is needed.

The present inventors have designed and successfully practiced a synthetic strategy for preparing isoindolinium salts, while employing various substituents for tuning steric hindrance, and have demonstrated their use in AEMs, while presenting enhanced stability in dry alkaline conditions.

Exemplary designed, synthesized and studied isoindolinium salts are shown in FIGS. 1B-C.

The newly designed isoindolinium salts can be easily prepared, and their chemical structure can be readily tuned. The newly designed isoindolinium salts were shown to perform at least similarly to, and even better than, commercially available membranes in terms of conductivity, power density and ASR, yet showed improved chemical stability. See, FIGS. 2, 3, 4A-B and 5A-B.

Some embodiments of the present invention therefore relate to novel isoindolinium salts (e.g., having Formula I as described herein), to processes of preparing same, to polymeric matrices comprising same and to their use as ion conductors in, for example, anion exchange membranes and in electrochemical devices containing same (e.g., fuel cells, batteries) and other articles-of-manufacturing.

In a search for additional solutions to limitations associated with currently practiced anion exchange membranes, the present inventors have designed and successfully practiced a new approach, which utilizes polyrotaxanes as non-covalent shields that kinetically hinder the hydroxide attack, and can be applied to ion conducting polymers (such as, but not limited to, QAs) in order to improve their chemical stability, particularly the chemical stability of anion conducting polymers in alkaline medium.

Embodiments of the present invention therefore further relate to the use of non-covalent shields, namely, a cyclic molecule mechanically interlocked with the polymer backbone, as schematically shown in FIGS. 6A and 6B, which “sits” around a cation (organic or inorganic) and doesn't affect it electronically, but “stands on the way” of hydroxide anions, inhibiting the decomposition reaction.

The large mechanically interlocked rings in polyrotaxanes offer additional advantages in terms of improving the chemical stability of AEMs, due to their large steric effect around the QAs in the main chain.

Reference is made to FIGS. 7A-B, which demonstrate the improved stability of AEMs containing polyrotaxanes, and to FIGS. 8 and 9 which provide further insights with regard to the polyrotaxane performance and the QA decomposition.

The herein described non-covalent shields (e.g., in a form of polyrotaxanes) can be used with any ion conducting polymer, bearing, for example, quaternary ammonium salt (QAs), sulfonium salts, phosphonium salts or inorganic cation in metallopolymers or in principle with any other cation.

The herein described non-covalent shields (e.g., in a form of polyrotaxanes) can be used with any cation molecule as well as an ionomeric cationic material that requires to be more stable in aggressive environment such as alkaline or oxidative conditions, etc., can be incorporated in an anion exchange membranes or any other solid electrolyte and in any electrochemical device where anion-exchange membranes or solid electrolytes are needed such as, for example, electrolyzers, AEMFCs, flow batteries, metal-air batteries, etc.

The herein described non-covalent shields (e.g., in a form of polyrotaxanes) are advantageous by offering a steric shielding effect without chemically altering the properties of the ionic conductor (e.g., a QA), by being general and complementary with any ion conductor and other components of, for example, AEM, and by providing kinetic rather than thermodynamic stabilization.

The herein described methodology acts as an “additive” which improves the chemical stability of all other functional groups in anion-exchange membranes. It can be relatively easily adopted and applied in existent polymer and membrane production lines. It may also have the advantage of imparting unique conductivity properties to the polymer/membrane, thanks to a potential “channeling” effect.

Embodiments of the present invention therefore further relate to a use of a polyrotaxane in forming solid electrolytes such as ion exchange membranes (e.g., anion exchange membranes), ion exchange membranes made thereof, and to electrochemical systems containing such solid electrolytes (e.g., fuel cells, batteries). The polyrotaxane can have a cyclic moiety associated with a polymer or a polymeric backbone. The cyclic moiety can be covalently attached to backbone units of a polymeric backbone, as pendant group(s) and/or terminal group(s), or non-covalently interacted with a polymer (e.g., mechanically interlocked around a polymer as described herein).

### Isoindolinium Salts

While many quaternary ammonium (QA) functional groups have been designed, prepared, and tested under harsh alkaline conditions in attempts to address the technological challenge of making functional groups that meet the commercial requirements for producing practical AEMs for AEMFCs, no QAs with sufficient lifetime have been found to date, especially in low hydration conditions where hydroxide ions are even more reactive.

The present inventors have considered the degradation mechanism of the currently most practiced quaternary ammonium salt (QA) in anion exchange membranes, BTMA (see, FIG. 1A), and have designed accordingly a new class of QAs, isoindolinium salts (see, FIG. 1B). The present inventors have demonstrated that the modification to the QA structure resulted in significant steric effects created by appropriate substituents on the benzylic carbons, thereby inhibiting its degradation and extending its lifetime.

The present inventors have uncovered that the QA functional group isoindolinium presents an extremely high stability in dry conditions, higher than BTMA (the currently known QA functional group that exhibit the best performance in most commercially available AEMs for use in AEMFC tests). See, for example, FIG. 2.

The newly designed QAs disclosed herein are modular, and can have numerous attachments sites for steric and electronic directing components, as shown in FIG. 1B, which can further improve the stability and/or other properties of the functional group, including its ion conductivity.

The newly designed isoindolinium-based QAs are structurally similar to BTMA, and therefore present many similarities and advantages. The process of connecting isoindolinium to polymeric matrices can be performed using methodologies already developed for BTMA. For example, isoindoline or N-substituted isoindolines can be directly reacted with halogen-functionalized membranes which present already low thickness and good mechanical properties. In addition, the structural similarity between isoindolinium and BTMA provides for similar ion conductivity. Isoindolinium, on the other hand, and contrary to BTMA, is almost planar, meaning that substitution at any of the positions marked in FIG. 1B can result in significant steric and electronic effects, that improve its stability towards hydroxide ion, as well as its capacity of ion conductivity.

As demonstrated in the Examples section that follows, studies were carried out with varying isoindolinium ions featuring a modular general structure as shown in FIG. 1B, which feature a myriad of substituents, including phenyl, methoxy, butoxy, t-butoxy, and bromo, as shown in FIG. 1C. Some of these compounds exhibited a significant improvement of stability compared to BTMA, at the harshest conditions of 0.1 water molecules per hydroxide.

The isoindolinium compounds can be connected to different polymeric backbones or matrices and utilized as ion conducting membranes in, for example, fuel cells and other electrochemical devices and articles-of-manufacturing, as described herein.

According to an aspect of some embodiments of the present invention there is provided an ion conductor comprising a polymeric matrix and a quaternary ammonium salt-containing moiety being is association with the polymeric matrix, the quaternary ammonium salt-containing moiety being an isoindolinium-containing moiety (also referred to herein as isoindolinium moiety).

By “being in association with” or “associated with” it is meant that the isoindolinium-containing moiety is linked to the polymeric matrix by means of physical or chemical interactions.

According to some of any of the embodiments described herein, the isoindolinium-containing moiety is covalently attached to the polymeric matrix. According to some of these embodiments, the polymeric matrix is a polymer, having a polymeric backbone composed of a plurality of backbone units, and the isoindolinium-containing moiety is or forms a part of pendant groups of a polymer or forms a part of the polymeric backbone, as described in further detail hereinafter.

According to embodiments of this aspect of the present invention, the quaternary ammonium salt-containing moiety is collectively represented by Formula I:

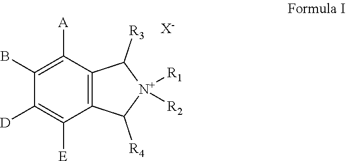

wherein:
R₁ and R₂ are each independently selected from alkyl, cycloalkyl and aryl, or represents an attachment point to the polymeric matrix;
X⁻is a counter anion;
R₃, R₄, A, B, D and E are each independently selected from hydrogen, alkyl, halo, haloalkyl, cycloalkyl, aryl, heteroaryl, hydroxy, alkoxy, aryloxy, and any other substituent as described herein, or represents an attachment point to the polymeric matrix.
According to some embodiments of the present invention, at least one of A and E is a bulky substituent capable of imparting steric hindrance around the positively charged portion of the compound.

According to some embodiments of the present invention, at least one of B and D is a bulky substituent that positions one or both of A and E at a configuration that imparts said steric hindrance.

According to some of any of the embodiments of the present invention, at least one of A and E is a bulky substituent capable of imparting steric hindrance around the positively charged portion of the compound, and at least one of B and D is a bulky substituent that positions one or both of A and E at a configuration that imparts said steric hindrance.

By “capable of imparting steric hindrance around the positively charges portion of the compound” it is meant that the substituent is sufficiently bulky (occupies a large space) so as to sterically mask the positive charge of the ammonium ion and its adjacent atoms, to thereby interfere with a possible interaction between the positive portion of the compound and other chemical species, for example, negatively charged species such as hydroxide ions. Thus, one or more of A and E substituents hinder the positively-charged portion and thereby protect it from, for example, an attack by negatively charged species such as hydroxide ions. Preferably, but not obligatory, one or both of the A and E substituents carry a partial negative charge or is electronegative and thereby provides also a repulsion of negatively charged species such as hydroxide ions, and/or interferes with an interaction between negatively-charged species and the positively-charged portion, and thus further protects the positively-charged portion of the compound from interacting with negatively-charged species such as hydroxide ions.

According to some embodiments, and as shown in FIG. 1B, the A and E substituents can provide steric and electronic effects that protect the positively-charged portion of the compound from interacting with negatively-charged species such as hydroxide ions.

By “a bulky substituent that positions one or both of A and E at a configuration that imparts said steric hindrance” with respect to the B and D substituents, it is meant that one or both of these substituents are sufficiently bulky and optionally carry a partial negative charge and thereby cause or force the adjacent substituents A and/or E to spatially arrange at a position that provides or enhances the steric hindrance of the positively charged portion of the compound discussed hereinabove.

According to some embodiments, and as shown in FIG. 1B, the B and D substituents can provide steric effects that result in protecting the positively-charged portion of the compound from interacting with negatively-charged species such as hydroxide ions.

According to some of any of the embodiments described herein, at least one of B and D is other than hydrogen or fluoro. Hydrogen and fluoro are examples of non-bulky substituents that do not affect the spatial arrangement of the A and/or E substituents.

According to some of any of the embodiments described herein, at least one of B and D is halo such as chloro, bromo, or iodo, preferably bromo or iodo (which are more bulky).

According to some of any of the embodiments described herein, at least one of B and D is an alkoxy, for example, methoxy, ethoxy, propoxy, isopropoxy, butoxy, t-butoxy, etc.

According to some of any of the embodiments described herein, at least one of A and E is alkoxy, for example, methoxy, ethoxy, propoxy, isopropoxy, butoxy, t-butoxy, etc.

According to some of any of the embodiments described herein, each of A and E is independently an alkoxy, for example, methoxy, ethoxy, propoxy, isopropoxy, butoxy, t-butoxy, etc.

According to some of any of the embodiments described herein, at least one or both of A and E is independently an aryl, for example phenyl.

According to some of any of the embodiments described herein, each of A and E is independently an alkoxy, and one or both of B and D is/are halo, for example, bromo. Exemplary such compounds are denoted as compounds 14 and 16 (see, FIG. 1C). As shown in FIG. 2, these compounds exhibit improved stability under alkaline conditions compared to BTMA.

According to some of any of the embodiments described herein, one of A and E is an alkoxy as described herein, and the other is halo, as described herein, for example, bromo. According to some of these embodiments, one or both of B and D is/are alkoxy, as described herein. An exemplary such compound is denoted herein as compound 17 (see, FIG. 1C).

According to some of any of the embodiments described herein, the ion conductor comprises a polymeric or co-polymeric backbone which comprises a plurality of backbone units, wherein at least a portion of the backbone units comprise the quaternary ammonium salt-containing moiety represented by Formula I.

The quaternary ammonium salt-containing moiety represented by Formula I, which is also referred to herein as an isoindolinium moiety, can be attached to the backbone units either covalently or non-covalently, e.g., by electrostatic interactions, hydrophobic interactions, pie-stacking interactions, etc. Preferably, the isoindolinium moiety is attached to the backbone units covalently, either directly, or via a linker.

According to some of any of the embodiments described herein, the quaternary ammonium salt-containing moiety represented by Formula I is covalently attached as a pendant group to a portion of the backbone units that compose the polymeric or co-polymeric backbone. The compound can be attached directly to the backbone units or via a linker. According to some of these embodiments, the polymer or co-polymer is composed of a plurality of backbone units, and at least a portion of the backbone units, e.g., 20%, 30%, 40%, 50%, 60%, 70%, 80%, 90% or even 100%, or any portion in a range of from 1% to 100% of the backbone units, has the indolinium moiety covalently attached thereto, either directly or indirectly via a linker.

According to some of these embodiments, each isoindolinium moiety is attached to a backbone unit via one attachment point. Alternatively, each isoindolinium moiety is attached to two backbone units via two attachment points.

In exemplary embodiments, the backbone is a co-polymeric backbone in which a portion of the backbone units bear a functional moiety to which the isoindolinium moiety can be covalently attached.

According to some of any of the embodiments described herein, the quaternary ammonium salt-containing moiety represented by Formula I forms a part of the polymeric backbone, that is, it is positioned between, and covalently linked to, two backbone units along the polymeric backbone. In such cases, a plurality of isoindolinium moieties is dispersed between the backbone units to form a co-polymeric backbone, and each isoindolinium moiety is attached to the two backbone units via two attachment points.

According to some of any of the embodiments described herein, an attachment point of the isoindolinium moiety to the backbone units in via one or two of A, B, D, E, R₁ or R₂. Alternatively, or in addition, the attachment point is via one or both of the carbon atoms adjacent to the nitrogen atom, denoted as R₃ and R₄ in Formula I.

According to exemplary embodiments, each isoindolinium moiety is attached to one or two backbone units via R₁ and/or R₂. In some of these exemplary embodiments, R₁ or R₂ Formula I represents an attachment point to the polymeric backbone.

According to exemplary embodiments, each isoindolinium moiety is attached to the backbone units via one of A, B, D or E, such that in Formula I, one of A, B, D and E represents an attachment point to a backbone unit of the polymeric backbone.

According to some of any of the embodiments of this aspect of the present invention, the attachment point to the backbone units can be directly, via a bond, or via a linker, or a linking moiety, such that when one or more of A, B, D, E, and R₁-R₄ is an attachment point, it can be a bond or a linking moiety as described herein. The linking can be, for example, an alkylene (e.g., methylene or ethylene), an ether (e.g., an alkylene glycol), or any other hydrocarbon moiety (e.g., of 1, 2, 3 or more carbon atoms) that can be interrupted by one or more heteroatoms, as defined herein.

According to some of any of the embodiments described herein, the polymeric or co-polymeric backbone comprises backbone units of one or more of polystyrene, polyethylene (PE), PTFE, polypropylene, polyethylene imide (PEI), polyimide (PI), poly(ethylenetetrafluoroethylene) (ETFE), polyether, epoxy polymer, poly(ethylene oxide) (PEO), poly(phenylene oxide) (PPO), polysulfone, poly(benzimidazole) (PBI) and poly(phenylene) (PP). Any other polymers and co-polymers that are usable, for example, for forming ion exchange membranes are contemplated.

According to an aspect of some embodiments of the present invention there is provided a method of synthesizing the quaternary ammonium salt-containing moiety represented by Formula I as described herein in any of the respective embodiments, the process being essentially as described and exemplified herein (see, for example, Example 1 in the Examples section that follows).

According to some embodiments, the method is effected by coupling a compound of Formula II:

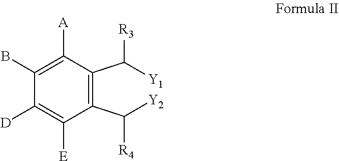

with a compound of Formula III:

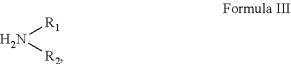

wherein A, B, D, E, R₁, R₂, R₃ and R₄ are as described herein for Formula I; and
Y₁ and Y₂ are each independently a leaving group.

In some embodiments, the coupling reaction is carried out in the presence of an alkali metal salt, MX, which generate the X⁻counter ion.

This synthetic pathway allows the preparation of a wide variety of isoindolinium salts by simply using selected starting materials of Formulae II and III, which bear the desired substituents.

The ion conductor comprising the isoindolinium salt attached thereto can be prepared by using a polymer or co-polymer that features reactive groups, for example, as pendant groups that are attached to at least a portion of the backbone units, that are capable of interacting with respective reactive groups or atoms of the isoindolinium salt, and coupling the polymer or co-polymer with the respective isoindolinium salt.

According to some embodiments, the polymer or co-polymer is a commercially available membrane that features a plurality of reactive groups as described herein, or in which such reactive groups are generated.

In exemplary embodiments, the coupling is effected between a polymer or co-polymer, or a membrane comprising same, having a plurality of pendant groups that feature a first reactive group, and an isoindolinium compound as described herein that features, in one or more of the positions that represent an attachment point to the polymer, a leaving group, or a linking moiety that terminates by a leaving group, such that the coupling is via a nucleophilic reaction of first reactive group.

In exemplary embodiments, the coupling is effected between a polymer or co-polymer, or a membrane comprising same, having a plurality of pendant groups that feature a first reactive group, and an isoindolinium compound as described herein that features, in one or more of the positions that represent an attachment point to the polymer, a second reactive group, or a linking moiety that comprises a second reactive group, such that the coupling is a reaction between the first and second reactive groups. The first and second reactive groups are selected capable of reacting with one another via, for example, nucleophilic reactions, addition-elimination reactions, click reactions, radical coupling, and any other coupling reactions, to thereby generate a respective linking moiety or bond.

### Anion Conducting Compositions

The present inventors have designed and successfully practiced a new approach of stabilizing the positive charge of QAs and other positively-charged ion conductors, and have demonstrated a considerable increase in the alkaline stability of such ion conductors.

As exemplified in FIGS. 6A and 6B, a linear polyquaternary ammonium salt was prepared and used to prepare four exemplary polyrotaxanes containing crown ethers of varying cavity size. Each polymer contains at least 30% and up to 150% crown ether molecules per polymeric QA. The alkaline stability of these polyrotaxanes and the control unthreaded polymer were evaluated and compared. As described in detail in the Examples section that follows, it was found that an increase in stability is seen and correlated to the size of the crown ether—bigger crown ethers lead to polyQAs with longer half-lives, indicating that the crown-ether works as a large steric shield, protecting the polyQA from adverse reactions with hydroxide ions.

The present inventors have demonstrated that this new approach increases the chemical stability of different functional groups in polymers, without tweaking their electronic properties, through a non-covalently bound steric shield.

According to an aspect of some embodiments of the present invention there is provided an anion conducting composition comprising an anion conducting polymer and a cyclic moiety associated therewith.

By “associated with” it is meant that the cyclic moiety is bound to the polymer via chemical or physical or mechanical interactions (e.g., mechanic interlocking as described herein).

The anion conducting polymer is typically comprised of a polymeric or co-polymeric backbone, composed of a plurality of backbone units, and a plurality of positively-charged moieties that are responsible for the anion transfer and hence for conducting anions. The positively charged moieties can form a part of the polymeric backbone or be attached to the polymeric backbone as pendant groups.

The anion conducting polymer can be a linear polymer, a cyclic polymer, or a hyperbranched polymer.

Exemplary anion conducting polymers include quaternary ammonium salt-containing polymer (QAs), including any of the QAs known in the art, for example, polymers bearing benzyl trimethylammonium groups, guanidinium groups, imidazolium groups, and isoindolinium groups such as described herein in any of the respective embodiments and any combination thereof. Additional exemplary anion conducting polymers include polymers bearing phosphonium groups (phosphonium-containing polymers), or sulfonium groups (sulfonium-containing polymer), and metallopolymers, such as for example, polymers bearing cobaltocenium. Any anion conducting polymer, particularly those usable in AEMs is contemplated.

According to some of any of the embodiments described herein, the anion conducting polymer is capable of conducting ions at a pH higher than 5, or higher than 6, or higher than 7, or higher (at alkaline pH).

According to some of any of the embodiments described herein, the anion conducting polymer is a quaternary ammonium salt-containing polymer, and in some of these embodiments it is a linear quaternary ammonium salt-containing polymer (QA).

According to some of any of the embodiments described herein, a mole ratio between the polymeric QA and the crown ether ranges from 10:1 to 1:10, or from 10:1 to 1:2, or from 10:1 to 1:1, or from 10:1 to 2:1, or from 10:1 to 5:1, including any intermediate values and subranges therebetween.

The cyclic moiety can be covalently attached to the polymer, for example, as linked to polymeric backbone units while forming a part of the backbone, or be attached as one or more pendant groups or as one or more terminal groups, to the polymeric backbone of the anion conducting polymer.

The cyclic moiety can alternatively be non-covalently attached to the ion conductor, for example, by means of mechanical interlocking as described in further detail herein.

According to some of any of the embodiments of this aspect of the present invention, the cyclic moiety is mechanically interlocked around a portion of the anion conducting polymer, as described herein. In some of these embodiments, the mechanical interlocking is by means of one or more end-capping moieties that form a part of the anion conducting polymer.

According to an aspect of some embodiments of the present invention there is provided an anion conducting composition comprising an anion conducting polymer featuring one or more end-capping moiety/ies at a backbone terminus thereof and/or at a pendant group terminus thereof and a cyclic moiety, the polymer, or a part thereof, is being threaded within the cyclic moiety. The end-capping moiety/ies each has a volume larger than a volume of the cyclic moiety (the volume of the inner cavity of the cyclic moiety) to thereby have the cyclic moiety mechanically interlocked around the polymer. In other words, the polymer is threaded within the cyclic moiety, in the cavity thereof, and remains threaded due to the end capping moieties that prevent from the cyclic moiety to unthread.

According to some of any of the embodiments of these aspects of the present invention, the polymer is a linear polymer.

According to some of any of the embodiments of these aspects of the present invention, the anion conducting polymer has a polymeric backbone composed of a plurality of backbone units, a portion or all of which can have pendant groups, and comprises end-capping moieties at each terminus of the polymeric backbone. In cases where the polymer is a linear polymer, it comprises two end-capping moieties, one at each terminus. In cases where the polymer is a branched or hyperbranched polymer, it may comprise an end-capping moiety at the terminus of each branch.

According to some embodiments of these aspects of the present invention there is provided an anion conducting composition comprising an anion conducting polymer featuring an end-capping moiety at each terminus thereof and a cyclic moiety, the polymer being threaded within a cyclic moiety, wherein each of the end-capping moieties has a volume larger than a volume of the (e.g., inner cavity of the) cyclic moiety to thereby have the cyclic moiety mechanically interlocked around the linear polymer.

According to some of these embodiments, the cyclic moiety is mechanically interlocked around the polymeric backbone, or, in other word, the polymeric backbone is threaded with the cyclic moiety.

According to some of these embodiments, the polymer is such that the anion conducting positively charged moiety forms a part of the polymeric backbone of the polymer. An exemplary such configuration is shown in FIG. 6B.

Alternatively, the polymer features a plurality (e.g., two or more) of pendant groups, and at least a portion, or all, of the pendant groups feature an end-capping moiety as described herein at the terminus thereof. In some of these embodiments, the cyclic moiety is mechanically interlocked around the pendant groups, by means of the end-capping moieties at one side and the polymeric backbone at the other side of the cyclic moiety.

According to some of these embodiments, the polymer is such that the anion conducting positively charged moiety forms a part of the pendant groups of the polymer. An exemplary such configuration is shown in Example 6 in the Examples section that follows.

According to some of any of the embodiments of this aspect of the present invention and any combination thereof, the cyclic moiety is a heterocyclic moiety.

According to some of any of the embodiments described herein, the heterocyclic moiety comprises at least one electronegative heteroatom. Without being bound by any particular theory, it is assumed that electronegative atom is in electrostatic interaction with the positively-charged portion of the anion conducting polymer.

According to some of any of the embodiments described herein, the cyclic moiety comprises at least 12, or at least 16, or preferably at least 18, carbon atoms and may optionally further comprise one or more heteroatoms such as oxygen, nitrogen, sulfur, etc.

According to some of any of the embodiments described herein, the cyclic moiety is a crown ether, for example, a crown ether featuring a ring of at least 12 carbon atoms, or at least 16 carbon atoms or at least 18 carbon atoms, and 6, 8 or 10 oxygen atoms, respectively.

The crown ether can be substituted or unsubstituted. In some embodiments, the crown ether comprises aromatic groups, as shown, as non-limiting examples in FIG. 6A.

The end-capping moiety or moieties can be selected in accordance with the size of the cyclic moiety, so as to feature a volume larger than the inner cavity of the cyclic moiety, to thereby provide the mechanical interlocking.

Exemplary end-capping moieties include, but are not limited to, aromatic or heteroaromatic moieties, featuring one, two or more rings, which can be fused to one another or be non-fused; and which can be substituted or unsubstituted; cycloalkyls or heteroalicyclic of at least 6 atoms, which can be substituted or unsubstituted; tertiary amine or quaternary ammonium groups, substituted by alkyls of at least 4 carbon atoms in length, and/or cycloalkyls of at least 6 carbon atoms, or aryls; each can independently be substituted or unsubstituted.

According to exemplary embodiments, the end-capping moiety is an aryl, for example, phenyl, which is substituted by two or more substituents. The substituents can be selected from alkyl, alkoxy, aryl, cycloalkyl, thioalkoxy, etc., as described herein.

As discussed and demonstrated herein, the association of the anion conducting polymer and the cyclic moiety provides for improved stability of the anion conducting polymer when subjected to an alkaline environment (e.g., of pH higher than 7, or higher than 8, or higher than 10, or higher). In some embodiments, the alkaline environment is a result of a presence of hydroxide ions which degrade the anion conducting polymer, as shown, as a non-limiting example, in FIG. 9 and further discussed in the Examples section that follows.

According to some of any of the embodiments described herein, a stability of the anion conducting composition when subjected to an alkaline environment as described herein is higher than a stability of the anion conducting polymer (in the absence of a cyclic moiety associated therewith as described herein).

According to some of any of the embodiments described herein, the cyclic moiety is for increasing a stability of the anion conducting polymer when subjected to alkaline environment.

According to some of any of the embodiments of these aspects of the present invention, the anion conducting polymer is an ion conductor that comprises an isoindolinium-containing moiety, as described herein in any of the respective embodiments and any combination thereof.

According to an aspect of some embodiments of the present invention there is provided an anion conducting composition comprising an ion conductor that comprises an isoindolinium-containing moiety as defined herein in any of the respective embodiments of this aspect of the present invention and any combination thereof, and a cyclic moiety associated with the ion conductor. The cyclic moiety can be covalently attached to the polymeric matrix, as described herein in any of the respective embodiments, for example, as linked to polymeric backbone units while forming a part of the backbone, or be attached as one or more pendant groups or as one or more terminal groups to a polymeric backbone to which the moiety of Formula I is attached, as described herein.

The cyclic moiety can alternatively be non-covalently attached to the ion conductor, for example, by means of mechanical interlocking as described herein in any of the respective embodiments.

According to an aspect of some embodiments of the present invention there is provided an anion conducting composition comprising an ion conductor that comprises an isoindolinium-containing moiety as described herein in any of the respective embodiments and any combination thereof, wherein the polymeric matrix comprises at least one end-capping moiety at a terminus thereof and/or at a pendant group terminus, ion conductor being threaded within a cyclic moiety, wherein the at least one end-capping moiety has a volume larger than a volume of the (e.g., inner cavity of) cyclic moiety to thereby have the cyclic moiety mechanically interlocked around the ion conductor, as described herein in any of the respective embodiments in the context of an anion conducting composition.

According to some of these embodiments, the polymeric matrix is a polymeric or copolymeric backbone to which the isoindolinium-containing moiety is attached by means of pendant groups, as described herein, and at least a portion of the pendant groups have an end-capping moiety as described herein in any of the respective embodiments at the free terminus thereof (not attached to the polymeric backbone), the end capping moiety forming together with the backbone mechanical interlocking. The pendant groups are threaded within the cyclic moiety.

According to alternative embodiments, the isoindolinium-containing moiety forms a part of the polymeric backbone, and the polymeric backbone has an end capping moiety as described herein in any of the respective embodiments at each terminus thereof, as described herein, such that the polymeric backbone is threaded within the cyclic moiety.

### Applications

Any of the ion conductors, and anion conducting compositions described herein feature a beneficial stability under alkaline environment and can therefore be beneficially utilized for forming an anion exchange membrane.

According to an aspect of some embodiments of the present invention there is provided an anion exchange membrane comprising an ion conductor that comprises an isoindolinium-containing moiety as described herein or an anion conducting composition comprising the ion conductor and a cyclic moiety as described herein in any of the respective embodiments.

According to an aspect of some embodiments of the present invention there is provided an anion exchange membrane comprising an anion conducting composition as described herein in any of the respective embodiments.

According to an aspect of some embodiments of the present invention there is provided an electrochemical system comprising the ion conductor or an anion conducting composition comprising same or an anion exchange membrane comprising the ion conductor or the anion conducting composition comprising the ion conductor, as described herein in any of the respective embodiments.

According to some of these embodiments, the electrochemical system is a fuel cell. According to an aspect of some embodiments of the present invention there is provided an electrochemical system comprising the anion conducting composition or the anion exchange membrane as described herein in any of the respective embodiments.

According to some of these embodiments, the electrochemical system is a fuel cell, such as an anion exchange membrane fuel cell (AEMFC).

According to some of any of the embodiments described herein, the anion exchange membrane and/or electrochemical system are characterized by water update, stability, current density, power density and any other performance parameter as demonstrated herein.

According to an aspect of some embodiments of the present invention there is provided an article-of-manufacturing comprising the ion conductor as described herein in any of the respective embodiments and any combination thereof or the ion (e.g., anion) exchange membrane as described herein in any of the respective embodiments and any combination thereof or the electrochemical system as described herein in any of the respective embodiments.

According to an aspect of some embodiments of the present invention there is provided an article-of-manufacturing comprising the anion conducting composition as described herein in any of the respective embodiments and any combination thereof or the anion exchange membrane as described herein in any of the respective embodiments and any combination thereof or the electrochemical system as described herein in any of the respective embodiments.

Exemplary articles-of-manufacturing include electrochemical devices such as fuel cells, batteries, electrolyzers, ionomers, ultracapacitors, ion and acid-base separators, as well as non-electrochemical articles such as disinfectants, antibacterial products, clothes, food packages, packaging of pharmaceutical or cosmetic products, anticoagulant-containing and/or antioxidant-containing pharmaceutical products.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein throughout, the phrase “linking group” or “linker” describes a group (a substituent) that is attached to another moiety in the compound or polymer via two or more atoms thereof. In order to differentiate a linking group from a substituent that is attached to another moiety in the compound via one atom thereof, the latter will be referred to herein and throughout as an “end group”.

As used herein, the term “amine” describes both a —NR′R″ end group and a —NR′— linking group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is or forms a part of a linking group.

The term “alkyl” describes a saturated aliphatic hydrocarbon including straight chain and branched chain groups. Preferably, the alkyl group has 1 to 20 carbon atoms. Whenever a numerical range; e.g. ,“1-20”, is stated herein, it implies that the group, in this case the alkyl group, may contain 1 carbon atom, 2 carbon atoms, 3 carbon atoms, etc., up to and including 20 carbon atoms. In some embodiments, the alkyl is a medium size alkyl having 1 to 10 carbon atoms. Unless otherwise indicated, the alkyl is a lower alkyl having 1 to 4 carbon atoms. In some embodiments, the alkyl has at least 4 carbon atoms, for example, the alkyl is having 4 to 12 or 4 to 10 or 4 to 8 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfinate, sulfate, sulfonate, sulfoxide, phosphonate, hydroxy, alkoxy, aryloxy, thiohydroxy, thioalkoxy, thioaryloxy, oxo, carbonyl, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain. When an alkyl is a linking group, it is also referred to herein as “alkylene”, e.g., methylene, ethylene, propylene, etc.

The term “alkenyl” describes an alkyl, as defined herein, in which at least one pair of carbon atoms are linked to one another via a double bond.

The term “alkynyl” or “alkyne” describes an alkyl, as defined herein, in which at least one pair of carbon atoms are linked to one another via a triple bond.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfinate, sulfate, sulfonate, sulfoxide, phosphonate, hydroxy, alkoxy, aryloxy, thiohydroxy, thioalkoxy, thioaryloxy, oxo, carbonyl, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfinate, sulfate, sulfonate, sulfoxide, phosphonate, hydroxy, alkoxy, aryloxy, thiohydroxy, thioalkoxy, thioaryloxy, oxo, carbonyl, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfinate, sulfate, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof. Preferably, the aryl is phenyl. Optionally, the aryl is naphthalenyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, triazine, tetrazine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfinate, sulfate, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof.

The term “alkaryl” describes an alkyl, as defined herein, which is substituted by one or more aryl or heteroaryl groups. An example of alkaryl is benzyl.

The term “halide” and “halo” describes fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═O)—O— group linking group, as these phrases are defined hereinabove, where R′ is as defined hereinabove.

The term “sulfinate” or “sulfinyl” describes a —S(═O)—OR′ end group or an —S(═O)—O— group linking group, as these phrases are defined hereinabove, where R′ is as defined hereinabove.

The term “sulfoxide” describes a —S(═O)R′ end group or an —S(═O)—O— linking group, as these phrases are defined hereinabove, where R′ is as defined hereinabove.

The term “sulfonate” or “sulfonyl” describes a —S(═O)₂—OR′ end group (also referred to herein as —SO₃R′ or —SO₃H) or an —O—S(═O)₂— linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein.

The term “N-sulfonamide” describes an R′S(═O)₂—NR″— end group or a —S(═O)₂—NR′— linking linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “phosphonate” describes a —P(═O)(OR′)(OR″) end group or a —P(═O)(OR′)(O)— linking group, as these phrases are defined hereinabove, with R′ and R″ as defined herein.

The term “thiophosphonate” describes a —P(═S)(OR′)(OR″) end group or a —p(═s)(or′)(O)— linking group, as these phrases are defined hereinabove, with R′ and R″ as defined herein.

The term “carbonyl” or “carbonate” or “ketone” as used herein, describes a —C(═O)—r′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, described a ═O end group.

The term “thiooxo” as used herein, described a ═S end group. The term “oxime” describes a ═N—OH end group or a ═N—O— linking group, as these phrases are defined hereinabove.

The term “hydroxyl” or “hydroxy” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” or “thio” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” or “nitrile” describes a —C≡N group.

The term “isocyanate” describes an —N═C═O group.

The term “nitro” describes an —NO₂ group.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)—O— linking group, as these phrases are defined hereinabove, where R′ is as defined herein.

The term “thiocarboxylate” as used herein encompasses “C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

The term “carbamate” as used herein encompasses N-carbamate and O-carbamate.

The term “N-carbamate” describes an R″OC(═O)—NR′R″— end group or a —OC(═O)—NR′— linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′— linking group, as these phrases are defined hereinabove, with R′ and R″ as defined herein.

The term “dithiocarbamate” as used herein encompasses N-dithiocarbamate and S-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

The term “guanyl” describes a R′R″NC(═N)— end group or a —R′NC(═N)— linking group, as these phrases are defined hereinabove, where R′ and R″ are as defined herein.

The term “guanidine” describes a —R′NC(═N)—NR″R′″ end group or a —R′NC(═N)—NR″— linking group, as these phrases are defined hereinabove, where R′, R″ and R′″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

As used herein, the term “alkylene glycol” describes a —O—[(CR′R″)_{z}—O]_{y}—R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo(alkylene glycol). Any of the compounds (e.g., active agents, compound of Formula I) described herein can be in a form of a pharmaceutically acceptable salt thereof.

As used herein, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms such as, for example, O, S, and/or amine. The number of carbon atoms can range from 2 to 30, and is preferably lower, e.g., from 1 to 10, or from 1 to 6, or from 1 to 4. A hydrocarbon can be a linking group or an end group.

Embodiments of the present invention encompass salts of any of the compounds or polymeric matrices as described herein.

Embodiments of the present invention further encompass solvates and hydrates of any of the substances described herein.

The term “solvate” refers to a complex of variable stoichiometry (e.g., di-, tri-, tetra-, penta-, hexa-, and so on), which is formed by a solute (the compound as described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water. The present embodiments further encompass any enantiomers and diastereomers of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

Commercially available materials and silica gel 60 (230-400 mesh) for column chromatography were obtained from Merck and used as received unless noted.

Thin-layer chromatography: silica gel GF254 plates were used and visualized with a UV lamp (254 nm).

NMR spectra were recorded in a Bruker Avance III 400 MHz or Bruker Avance 300 MHz spectrometer. The chemical shifts are referenced to signal at δ=0.00 ppm (TMS) or partially undeuterated solvent peaks.

Preparation of water-free hydroxide solution: In a three-neck flask, 18-crown-6 (CE) is heated to 60° C. under argon. Potassium metal was added to the flask, providing a blue solution. Small and precise amounts of Milli-Q water were injected into the potassium solution, until color changed to brown, indicating the end of the potassium titration. This mixture was then cooled to room temperature and stored in a glove box. 0.5 M CE-KOH in DMSO was prepared by dissolving 226 mg of a CE-KOH mixture (prepared as previously described [Dekel et al., Chem. Mater. 2017, 29 (10), 4425-4431], stored and handled in an MBraun Unilab 1200 glovebox) in 0.5 mL of dry DMSO. If hydrated hydroxide is desired for the test, adequate quantity of water was added instead of DMSO. In an NMR tube, QA salt (0.035 mmol) was dissolved in dry DMSO-d₆ (0.1 ml). The KOH solution was then added to the NMR tube and sealed with a rubber septum, Teflon, and parafilm. Mesitylene (1.6 μl) was added as an internal standard. After the injection, the tube was manually mixed and inserted into the NMR instrument.

### Stability Tests

It is well-known that water plays a major role in the properties of AEMs. A recently developed ex-situ method for measuring the chemical stability of AEMs in a fuel cell-like environment [Müller et al. ACS Mater. Lett. 2020, 2 (2), 168-173] can be used to evaluate the membrane stability. In comparison with all current methods to measure chemical degradation of AEMs, this technique offers reliability and reproducibility, as no liquid electrolyte (with all the involved complications) is used at any time of the test. This also assures to mimic the in-operando fuel cell environment, measuring then the real stability behavior of an AEM during fuel cell operation. Stability tests presenting percentage of QA remaining control polymer and compound of interest (e.g., isoindolinium salts in FIG. 2 and polyrotaxanes in FIGS. 7A and 7B) as a function of time during reaction with 0.5 M OH⁻ in DMSO-d₆ at room temperature, as determined by NMR data. This procedure was previously described in Dekel et al., 2017 supra.

### Membrane Characterization

Membranes are further characterized by determining ion-exchange capacity (IEC); water uptake (WU); ion conductivity; mechanical testing and water swelling ratio, as follows.

### IEC

IEC was determined by chloride titration using a Metrohm titrator based on a previously described procedure (for example, Zheng et al. Macromolecules, 2018, 51 (9), 3264-3278). The membrane was immersed into a KC1 solution (1.0 M) for 48 hours. Then, it was washed by soaking in deionized water for 48 hours. Finally, the membrane was in immersed into a KNO₃ solution (1.0 M) for 48 hours, after which this solution was titrated with AgNO₃ (0.01 M) to determine the free Cl⁻ concentration, using an Ag electrode. IEC is expressed as millimoles of Cl⁻ per gram of dry membrane (mmol/gram), according to the following formula:

$$IEC ( \frac{mmol}{gram} ) = \frac{Δ {V}_{{AgNO}_{3}} \times {C}_{{AgNO}_{3}}}{W ( dry )}$$

### Water Uptake Measurements

The WU was measured using a VTI SA⁺ instrument (TA Instruments, USA). The relative humidity (RH) was determined with a two-stage chilled-mirror dew-point analyzer and adjusted by mixing dry and humidified nitrogen gas. Each sample was initially dried in situ for up to 150 minutes at 60° C. and RH of about 0%. Then, the temperature was decreased to 25° C. and 40° C., and a 95% RH step-increase is done. The RH was maintained until sample weight reached equilibrium (<0.001 wt % change in 5 minutes). WU values are calculated from the “wet” weight [W(wet)] at the end of the initial drying step, according to the formula:

$$\% WU = \frac{W ( wet ) {-}^{36} W ( dry )}{W ( dry )} \times 100 \%$$

The WU kinetics was measured by following the mass change of the AEM as a function of time as a result of the applied RH step. The characteristic time constant, τ, was calculated by fitting the experimental data to the following equation:

$$\frac{{W}_{t} - {W}_{0}}{{W}_{∞} - {W}_{0}} = \frac{{M}_{t}}{{M}_{∞}} ≅ 1 - exp ( - \frac{t}{τ} )$$

### Ion Conductivity

Ion conductivity test was performed by a previously described procedure [see Ziv, N. and Dekel, D. R. Electrochem. commun. 2018, 88, 109-113].

### Fuel Cell Test

Membranes are further evaluated by AEMFCs tests. These include optimization of fuel cell assembly. Initially, polarization curves are done at different temperatures to characterize performance (power and limiting current density) and operational stability as a function of temperature.

In exemplary assays, voltage, ASR and power density were measured vs. current density at cell temperature of 60° C. with optimized dew points and 100 kPa back-pressurization on both anode and cathode; and of 50 and 40° C. with optimized dew points and 0 kPa back-pressurization.

In other exemplary assays, long term stability tests were performed at 60° C. under a constant current density of 300 mA/cm², with 100 kPa back-pressurization on both anode and cathode.

More specifically, gas diffusion electrodes with an active area size of 5 square centimeter (cm²) each were prepared for the anode and cathode, following the previously reported general procedure [see for example, Douglin et al. J. Power Sources Adv. 2020, 5, 100023]. The anode and cathode were loaded to 0.55 mgp_{PtRu}/cm² and 0.52 mg_{Pt}/cm², respectively. The electrodes along with a 9 cm 2 piece of the AEMFC membrane were immersed in aqueous 1 M KOH solution for 1 hour, with solution changes every 20 minutes, to convert to hydroxide form. The AEMFC was then assembled in-situ between two 5 cm² single-serpentine graphite bipolar flow field plates and pressed using a 4.5 N m torque. The AEMFC was tested in an 850E Scribner Associates Fuel Cell test station by first heating to 40° C. while flowing N₂ at 0.5 slpm until the temperature stabilized. Then, pure humidified H₂ and O₂ reactant gases were flowed at 1 slpm without back-pressurization. After the acquisition of polarization curves, the temperature was increased to 50 and 60° C., dew points optimized and 100 kPa back-pressurization on both anode and cathode applied at 60° C. only before the acquiring other polarization curves at those temperatures. Immediately after the 60° C. polarization curve, the cell was subjected to stability testing under the same operating conditions and a constant load of 300 mA/cm².

### Example 1

### Syntheses of Exemplary Isoindolinium Salts

The present inventors have studied the use of an isoindolinium salt in AEMFC, and particularly the chemical stability thereof under dry alkaline conditions.

The basic isoindolinium (Compound 11; see, FIGS. 1B and 1C) features structural features similar to benzyltrimethylammonium (BTMA) (see, FIG. 1A), but has a cyclized ammonium salt, putting the ortho positions of the aromatic ring on the pathway for hydroxide attack.

The present inventors have studied the effect of varying substituents on the chemical stability of the isoindolinium salt, and to this end have synthesized, using two synthetic approaches as described in detail hereinafter, exemplary substituted isoindolinium compounds, the structures of which are shown in FIGS. 1B and 1C.

### Synthesis of QA1 (Compound 11)

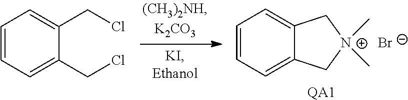

In a 100 mL round bottom flask, 1,2-bis(chloromethyl)benzene (554 mg, 3.16 mmol) were dissolved in ethanol (10 mL) and then K₂CO₃ (437 mg, 3.16 mmol), N,N-dimethylamine (0.4 mL of a 40% aqueous solution, 3.16 mmol) and KI (52 mg, 0.316 mmol) were added. The reaction mixture was stirred for 12 hours at room temperature. The solvent was removed under reduced pressure and the obtained solid was washed with diethyl ether and dissolved in water. Thereafter, the ammonium salt was precipitated using saturated solution of NH4PF6 to provide the product as a white solid. Yield 780 mg, 92%.

¹H NMR (400 MHz, CD₃CN): δ=7.44 (t, 2H), 7.43 (d, 2H), 4.77 (s, 1H), 3.25 (s, 1H) ppm.

¹³C NMR (101 MHz, CD₃CN): δ=134.09, 130.22, 124.60, 71.54, 53.58 ppm. HRMS (ESI) m/z 148.1107 (calculated C₁₀H₁₄N⁺, 148.1121).

### Synthesis of QA2 (Compound 12)

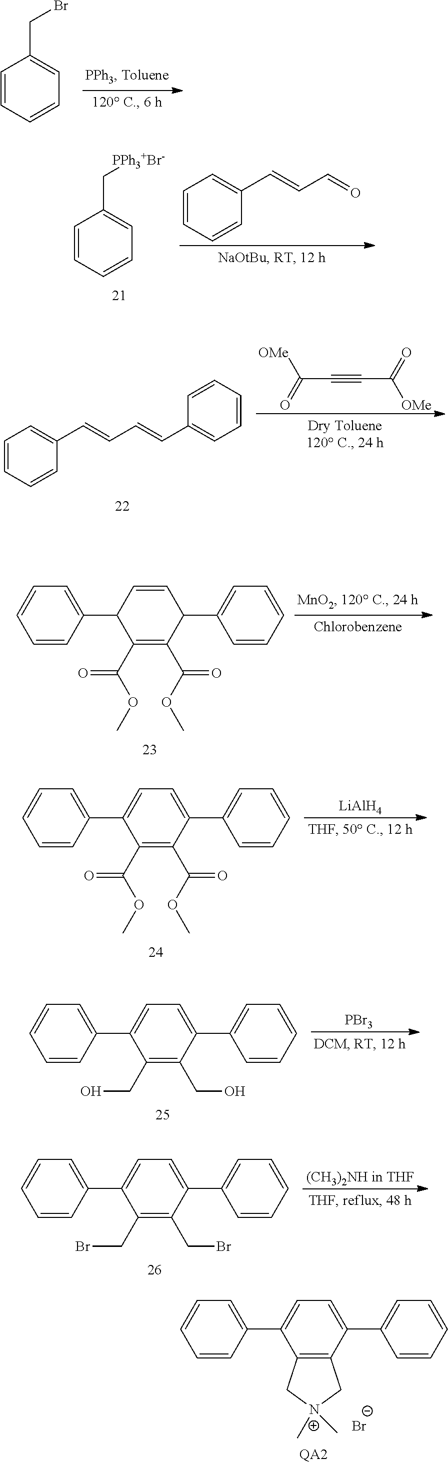

Synthesis of 21: In a 100 mL round bottom flask, triphenylphosphine (4.6 grams, 17.54 mmol) was dissolved in toluene (50 mL) and then, benzyl bromide (1.4 mL, 11.69 mmol) was added. The reaction mixture was stirred at 120° C. for three hours. The reaction was thereafter cooled and the white precipitate was filtered, washed with cold diethyl ether and dried in air to provide benzyl triphenyl phosphonium bromide 21 as a fine white crystalline solid used directly for the next step. Yield 4.08 grams, 80%. Structures was verified by NMR in comparison to the literature [Wen et al. Arkivoc, 2005, 2005, 169].

Synthesis of 22: In a 250 mL round bottom flask, 21 (3 grams, 6.92 mmol) was dissolved in tetrahydrofuran (THF; 100 mL) and cooled to 0° C. To this, potassium tert-butoxide (0.93 gram, 8.30 mmol) was added while stirring vigorously. After 20 minutes, a clear solution was obtained. Trans-cinnamaldehyde (0.7 mL, 5.8 mmol) dissolved in THF (10 mL) was then added dropwise. Once the addition was complete, the reaction was allowed to warm to room temperature, and stirred for 12 hours. The solvent was evaporated under reduced pressure and the solid re-dissolved in ethyl acetate (200 mL). The solution was then washed with water (2×50 mL), dried over anhydrous Na₂SO₄ and evaporated. The solid obtained was recrystallized from ethanol, providing the product 22 as a white crystalline solid. Yield 1.10 gram, 77%. Structure was verified by NMR in comparison to literature [Yasukawa et al., Green Chem., 2018, 20, 1213].

Synthesis of 23: In a 50 mL round bottom flask, 22 (3 grams, 14.5 mmol) was dissolved in dry toluene (15 mL) under argon. To this, dimethyl but-2-ynedioate (2.15 mL, 15.9 mmol) was added. The reaction mixture was refluxed for 20 hours. The solvent was evaporated under reduced pressure and the obtained solid washed with hexane, to provide 23 as a white solid. Yield 4.05 grams, 80%. Structures was verified by NMR in comparison to literature (Yasukawa et al., 2018, supra).

Synthesis of 24: In a 100 mL round bottom flask, 23 (4 grams, 11.5 mmol) was dissolved in chlorobenzene (30 mL) and MnO₂ (5.2 grams, 57.4 mmol) was added. The reaction mixture was stirred at 120° C. for 24 hours. The reaction mixture was filtered through Celite®, followed by CH₂Cl₂ washing, and the combined filtrate was evaporated under reduced pressure. Compound 24 was obtained as an off-white solid. Yield 3.58 grams, 90%. Structures was verified by NMR in comparison to literature [Li et al., Org. Lett., 2017, 19, 4078].

Synthesis of 25: In a 100 mL round bottom flask, 24 (1.2 gram, 3.468 mmol) was dissolved in dry THF (20 mL) and cooled to at 0° C. LiAlH₄ (0.53 gram, 13.8 mmol) was added in small portions, under N₂ flow. The reaction mixture was stirred for 12 hours at 50° C. The reaction was quenched by ice-cold water and then EtOAc (10 mL) was added. The phases were separated, the organic phase was filtered, dried over Na2SO4 and concentrated under reduced pressure, providing 25 as a pale-yellow solid. Yield 0.81 mg, 81%. Structures was verified by NMR in comparison to literature [Yasukawa et al., Green Chem., 2018, 20, 1213].

Synthesis of 26: In a glove box, 25 (2 grams, 6.89 mmol) was dissolved in dry dichloromethane (DCM; 50 mL) in a 100 mL round bottom flask, and PBr₃ (0.65 mL, 6.89 mmol) was added dropwise at ambient temperature. The reaction mixture was stirred for 12 hours. The solvent was thereafter evaporated under reduced pressure and the solid residue was washed with acetone. All the by-products were soluble in acetone and the remainder white color solid was pure compound 26. Yield 2.4 grams, 84%. Structure was verified by NMR in comparison to literature [Geng et al., Chem. Mater., 2002, 14, 46].

Synthesis of QA2 (Compound 12): In a 50 mL round bottom flask, 26 (0.5 gram, 1.2 mmol) was dissolved in THF (10 mL). To this, N,N-dimethylamine (2.0 M in THF, 65 μL, 1.2 mmol) and K₂CO₃ (333 mg, 2.4 mmol) were added. The reaction mixture was refluxed for 48 hours. The solvent was removed under reduced pressure and the solid obtained was re-dissolved in CHCl₃ (10 mL) and filtered. The filtrate was collected and evaporated. The desired product QA2 was obtained as a white solid. Yield 320 mg, 71%.

¹H NMR (400 MHz, DMSO-d₆): δ=3.24 (s, 6H), 5.11 (s, 4H), 7.49-7.59 (m, 10H), 7.67 (s, 2H) ppm.

¹³C NMR (101 MHz, DMSO-d₆): δ=52.0, 72.0, 128.0, 128.2, 128.5, 129.1, 129.5, 129.6, 132.0, 136.1, 137.9 ppm.

HRMS (ESI): m/z=300.1758 (calculated C₂₂H₂₂N⁺, 300.1747).

### Synthesis of QA3 (Compound 13)

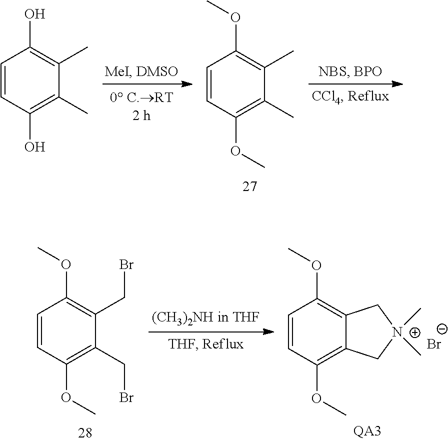

Synthesis of 27: 2,3-dimethyl-hydroquinone (10 grams, 72.38 mmol) and potassium hydroxide (20.30 grams, 361.89 mmol) were added to a 500 mL round bottom flask, and DMSO (200 mL) was added. The reaction mixture was stirred at room temperature for 30 minutes. Then, the reaction mixture was cooled to 0° C., and methyl iodide (11.5 mL, 180.95 mmol) was added for 1.5 hours. Water (200 mL) was added to the reaction and extracted with EtOAc (3×150 mL). The combined organic phase was dried over anhydrous Na₂SO₄ and evaporated. Compound 27 was obtained as a crystalline red-brown solid. Yield 7.48 grams, 90%. Structures was verified by NMR in comparison to literature [Bedford et al., Tetrahedron, 2013, 69, 4389].

Synthesis of 28: In a 50 mL round-bottom flask, 27 (1 gram, 6.02 mmol) was dissolved in CCl₄ (20 mL). Then, freshly recrystallized N-bromosuccinimide (NBS; 2.25 grams, 12.63 mmol) and benzoyl peroxide (29.16 milligrams, 0.12 mmol) were added and the reaction mixture was refluxed for 16 hours. The solvent was evaporated under reduced pressure and the remaining solid re-dissolved in CHCl₃ (100 mL). The solution was washed with water (2×25 mL), dried over anhydrous Na₂SO₄ and evaporated. The solid obtained was recrystallized using methanol to provide the pure compound 28 as a white solid. Yield 917 milligrams, 47%. Structure was verified by NMR in comparison to literature [Mazzini et al., European J. Org. Chem. 2006, 2006, 5588].

### Synthesis of QA3 (Compound 13)

In a 50 mL round bottom flask, 28 (0.5 gram, 1.54 mmol) was dissolved in THF (10 mL). To this, N,N-dimethylamine (2.0 M in THF, 81 μL, 1.54 mmol) and K₂CO₃ (424 mg, 3.08 mmol) were added. The reaction mixture was refluxed for 48 hours. The solvent was removed under reduced pressure and the solid obtained was re-dissolved in CHCl₃ (10 mL) and filtered. The filtrate was collected and evaporated. The product QA3 was purified by recrystallization in methanol to provide a pure white solid. Yield 179 mg, 56%.

¹H NMR (400 MHz, CDCl₃): δ=3.77 (s, 6H), 3.80 (s, 6H), 4.99 (s, 4H), 6.80 (s, 2H) ppm.

¹³C NMR (101 MHz, CDCl₃): δ=53.87, 55.89, 69.94, 112.36, 121.61, 149.06 ppm.

HRMS (ESI): m/z=208.1313 (calculated C₁₂H₁₈NO₂⁺, 208.1338).

### Synthesis of QA4 (Compound 14)

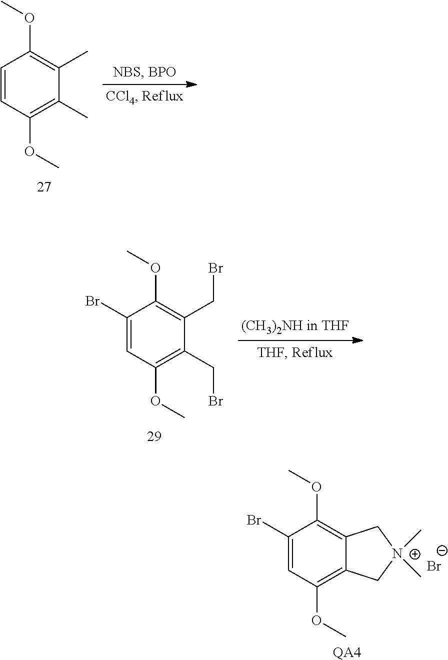

Synthesis of 29: In a 50 mL round-bottom flask, 27 (1 gram, 6.02 mmol) was dissolved in CCl₄ (10 mL). Then, freshly recrystallized NBS (3.54 grams, 19.87 mmol) and benzoyl peroxide (46 milligrams, 0.19 mmol) were added and the reaction mixture was refluxed for 16 hours. The solvent was evaporated under reduced pressure and the remaining solid re-dissolved in CHCl₃ (100 mL). The solution was washed with water (2×25 mL), dried over anhydrous Na₂SO₄ and evaporated, providing 29 as a yellow oil. Yield 1.04 gram, 40%. Structure was verified by NMR in comparison to literature [Rinke et al., Pharmazie, 2001, 56, 763].

### Synthesis of QA4 (Compound 14)

In a 50 mL round-bottom flask, 29 (1 gram, 2.48 mmol) was dissolved in THF (10 mL). To this, N,N-dimethylamine (2.0 M in THF, 128 μL, 2.45 mmol) and K₂CO₃ (678 mg, 4.90 mmol) were added. The reaction mixture was refluxed for 48 hours. The solvent was then removed under reduced pressure and the solid obtained was re-dissolved in CHCl₃ and filtered. The filtrate was collected and evaporated. The product was purified by recrystallization in methanol, providing pure QA4 as a yellow oil. Yield 730 mg, 80%.

¹H NMR (400 MHz, CD₃CN): δ=3.29 (s, 6H), 3.77 (s, 3H), 3.82 (s, 3H), 4.67 (s, 2H), 4.93 (s, 2H), 7.22 (s, 1H) ppm.

^{13 C NMR (}101 MHz, CD₃CN): δ=54.29, 57.30, 61.71, 69.64, 70.04, 117.71, 118.00, 122.58, 128.42, 147.00, 152.29 ppm.

HRMS (ESI): m/z=272.0256 (calculated [M−CH₃]⁺; C₁₁H₁₅BrNO₂⁺, 272.0281).

### Synthesis of QA5 (Compound 15)

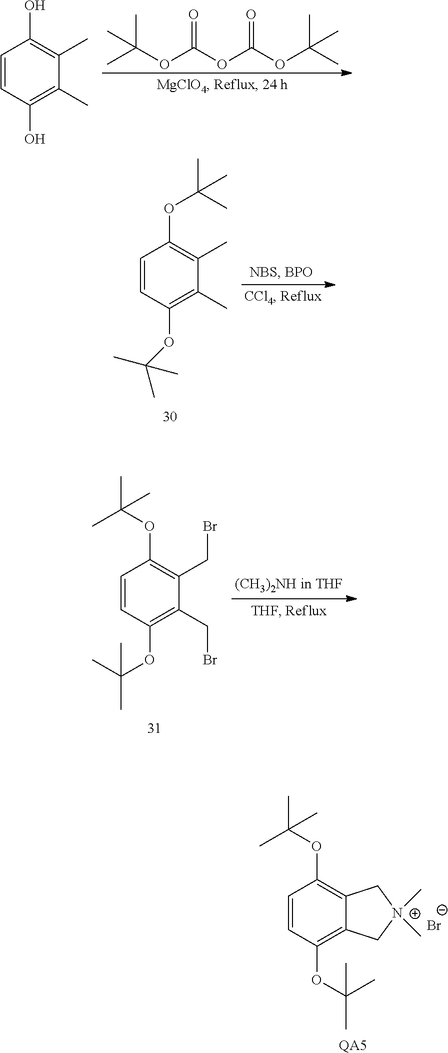

Synthesis of 30: 2,3-Dimethyl-hydroquinone (6.91 grams, 50.0 mmol) was added to a 250 mL round bottom flask. Magnesium perchlorate (2.23 grams, 10.0 mmol) and Boc₂O (110 mL, 500 mmol) were added (rapid gas evolution occurred for several minutes). The reaction mixture was refluxed for 24 hours, and then cooled, quenched by H₂O (100 mL), neutralized by aq. NaOH and extracted with ethyl acetate (3×100 mL). The combined organic layers were washed with water (100 mL) and brine (100 mL), dried over Na₂SO₄ and evaporated. The crude material was purified via flash column chromatography (silica gel, hexane/ethyl acetate=98/2) to provide product 30 as a yellow oil. Yield 8.75 grams, 70%. Structure was verified by NMR in comparison to literature [Heilmann and Tiefenbacher, Chem.-A Eur. J., 2019, 25, 12900].

Synthesis of 31: In a 50 mL round-bottom flask, 30 (1 gram, 3.99 mmol) was dissolved in CCl₄ (20 mL). Then, freshly recrystallized NBS (1.50 gram, 8.38 mmol) and benzoyl peroxide (19.37 milligrams, 0.08 mmol) were added and the reaction mixture was refluxed for 16 hours. The solvent was evaporated under reduced pressure and the remaining solid re-dissolved in CHCl₃ (100 mL). The solution was washed with water (2×25 mL), dried over anhydrous Na₂SO₄ and evaporated, providing 31 as a yellow oil. Yield 1.46 gram, 90%. Structure was verified by NMR in comparison to literature [Heilmann and Tiefenbacher, Chem.-A Eur. J., 2019, 25, 12900].

### Synthesis of QA5 (Compound 15)

In a 50 mL round-bottom flask, 31 (1 gram, 3.08 mmol) was dissolved in THF (10 mL). To this, N,N-dimethylamine (2.0 M in THF, 163 μL, 3.08 mmol) and K₂CO₃ (850 milligrams, 6.17 mmol) were added. The reaction mixture was refluxed for 48 hours. The solvent was then removed under reduced pressure and the solid obtained was re-dissolved in CHCl₃ and filtered. The filtrate was collected and evaporated. The product was purified by recrystallization in methanol, providing pure QA5 as a yellow solid. Yield 573 milligrams, 80%.

¹H NMR (400 MHz, CDCl₃): δ=1.31 (s, 18H), 3.28 (s, 6H), 4.84 (s, 4H), 7.02 (s, 2H).

¹³C NMR (101 MHz, CDCl₃): δ=29.08, 53.20, 7.019, 80.80, 124.17, 127.54.

HRMS (ESI): m/z=292.2265 (calculated C₁₈H₃₀NO₂⁺, 292.2277).

### Synthesis of QA6 (Compound 16)

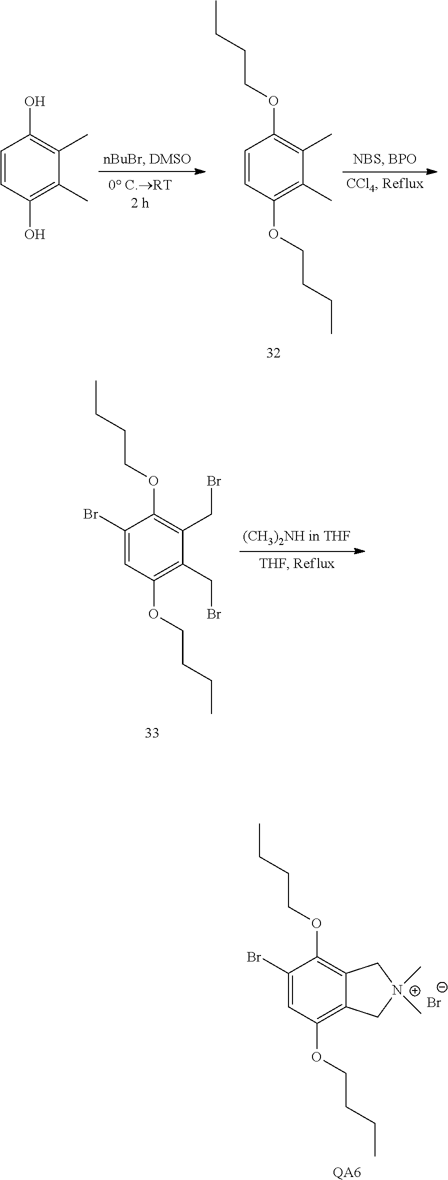

Synthesis of 32: In a 100 mL round-bottom flask, 2,3-dimethyl hydroquinone (1 gram, 7.23 mmol) was dissolved in 40 mL of DMSO and to this potassium hydroxide (2.03 grams, 36.18 mmol) was added. The reaction mixture was stirred at room temperature for 0.5 hour. After cooling down the reaction mixture to 0° C., n-butyl bromide (1.72 mL, 15.1 mmol) was added for 0.5 hour. The completion of reaction was confirmed by thin layer chromatography (TLC) and the reaction was quenched by adding water. The reaction mixture was extracted with ethyl acetate (3×150 mL), dried with anhydrous Na₂SO₄ and evaporated. Yellow color solid was obtained as product 32. Yield 1.63 gram, 90%.

¹H NMR (101 MHz, CDCl₃): δ=0.97-1.01 (t, 6H), 1.50-1.55 (m, 4H), 1.74-1.80 (m, 4H), 2.19 (s, 6H), 3.89-3.92 (t, 4H), 6.65 (s, 2H).

¹³C NMR (101 MHz, CDCl₃): δ=12.26, 144.05, 19.57, 31.82, 68.79, 109.37, 127.13, 151.42.

HRMS (ESI): m/z=251.1966 (calculated (C₁₆H₂₇O₂)⁺, 251.1988).

Synthesis of 33: In a 100 mL round bottom flask 32 (1 gram, 2.45 mmol) was dissolved in 12 mL of CCl₄ and to this freshly recrystallized NBS (1.44 gram, 8.08 mmol) and benzoyl peroxide (19.37 mg, 0.08 mmol) was added. The reaction mixture was refluxed (at about 95° C.) for 16 hours. The completion of reaction was confirmed by ¹H NMR, the solvent was evaporated under reduced pressure and re-dissolved in CHCl₃ (100 mL). The organic layer was washed with water (25 mL×2), dried with anhydrous Na₂SO₄ and evaporated. Yellow oil is obtained as 33. Yield 1.65 gram, 85%.

¹H NMR (400 MHz, CDCl₃): δ=0.98-1.04 (m, 6H), 1.49-1.59 (m, 4H), 1.61-1.87 (m, 4H), 3.97-4.00 (t, 2H), 4.03-4.07 (t, 2H), 4.71 (s, 1H), 4.72 (s, 1H), 7.04 (s, 1H) ppm.

¹³C NMR (101 MHz, CDCl₃): δ=13.84, 14.03, 19.20, 19.30, 23.53, 23.73, 31.23, 32.19, 68.86, 74.00, 117.30, 118.47, 125.99, 132.80, 148.71, 153.84 ppm.

HRMS (ESI): m/z=485.9228 and 487.9207 (calculated C₁₆H₂₃Br₃O₂⁺, 485.9206 and 487.9183).

### Synthesis of QA6 (Compound 16)

In a 50 mL round bottom flask, 33 (1 gram, 2.05 mmol) was dissolved in chloroform (10 mL). To this dimethylamine (2.0 M in THF, 100 μL, 2.46 mmol) and K₂CO₃ (848 mg, 6.18 mmol) was added. The reaction mixture was refluxed for 24 hours and monitored by ¹H NMR. The reaction mixture was filtered and the filtrate was evaporated under vacuum. Yellow oil obtained was dissolved in minimum amount of dichloromethane and precipitated in n-pentane and washed with diethyl ether. Yellow color solid was obtained as product QA6. Yield 419 mg, 70%.

¹H NMR (400 MHz, CDCl₃): δ=0.92-0.96 (m, 6H), 1.39-1.51 (m, 4H), 1.68-1.76 (m, 4H), 3.78 (s, 6H), 3.89-3.93 (m, 4H), 4.93 (s, 2H), 5.24 (s, 2H), 7.01 (s, 1H) ppm.

¹³C NMR (101 MHz, CDCl₃): δ=53.58, 68.92, 69.24, 69.49, 74.47, 117.40, 118.30, 120.82, 127.43, 145.58, 150.72 ppm.

HRMS (ESI): m/z=370.1377 and 372.1356 (calculated C₁₈H₂₉BrNO₂⁺, 370.1404 and 372.1390).

### Synthesis of QA7 (Compound 17)

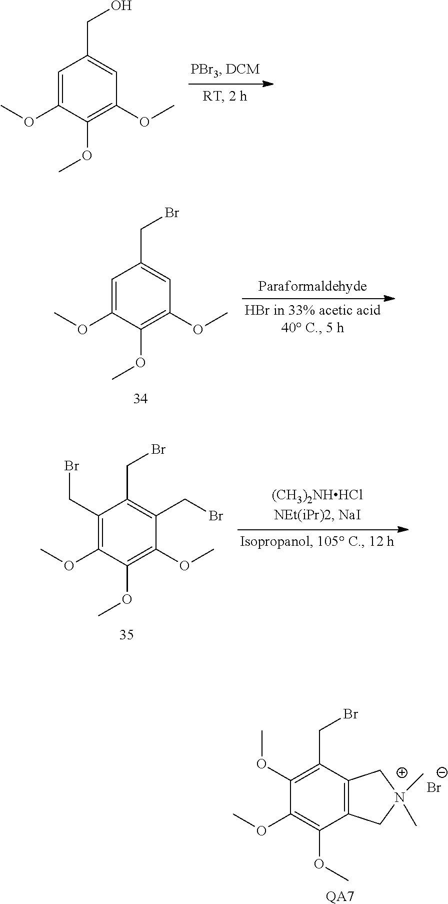

Synthesis of 34: In a 100 mL round bottom flask, 3,4,5-trimethoxy benzyl alcohol (1 gram, 5.044 mmol) was dissolved in dichloromethane (50 mL) and then phosphorus tribromide (0.23 mL, 2.52 mmol) was added at 0° C. The reaction mixture was stirred at room temperature for one hour. The reaction was cooled to 0° C., water (100 mL) was added very slowly. The organic layer was collected and washed with water (2×50 mL), dried over anhydrous Na₂SO₄, and evaporated. White crystalline solid was obtained as product 34. Yield 1.18 gram, 90%.

¹H NMR (400 MHz, CDCl₃) δ [ppm]=6.54 (s, 1H), 4.39 (s, 1H), 3.79 (s, 3H), 3.77 (s, 1H) ppm.

¹³C NMR (101 MHz, CDCl₃) δ [ppm]=153.3, 138.13, 133.2, 106.1, 60.9, 56.2, 34.4 ppm.

Synthesis of 35: To a mixture of 34 (1 gram, 3.85 mol) and paraformaldehyde (0.23 gram, 15 7.70 mol), HBr (33% in acetic acid, 10 ml) was added dropwise. The mixture was stirred at 40° C. for 2 hours before water (250 mL) was added and the mixture was extracted with dichloromethane (2×100 mL). The combined organic layers were washed with brine (2×50 mL), the organic phase was dried over anhydrous Na₂SO₄ and evaporated. Recrystallization from ethanol provided a white color solid was as product 35. Yield 1.54 gram, 90%.

¹H NMR (400 MHz, CDCl₃) δ [ppm]=4.67 (s, 2H), 4.61 (s, 4H), 3.94 (s, 6H), 3.82 (s, 3H) ppm.

¹³C NMR (101 MHz, CDCl₃) δ [ppm]=152.5, 145.6, 130.8, 126.2, 60.3, 59.6, 24.2, 22.8 ppm.

### Synthesis of QA7 (Compound 17)

In a 10 mL pressure flask, compound 35 was dissolved in isopropanol. Then, dimethylamine hydrochloride (82 mg, 1.00 mmol), DIPEA (0.34 mL, 1.95 mmol) and sodium iodide (68 mg, 0.46 mmol) were added and the reaction stirred at 105° C. for 15 hours. The reaction was then cooled, filtered and the filtrate was evaporated. The residue obtained was re-dissolved in dichloromethane (100 mL) and extracted with water (3×100 mL). The combined organic layers were washed with sodium bicarbonate (2×100 mL), dried over anhydrous Na₂SO₄ and evaporated. Yellow color solid was as product QA7. Yield 150 milligram, 36%.

¹H NMR (400 MHz, CD₃CN): δ=5.60 (s, 2H), 4.98 (s, 2H), 4.73 (s, 2H), 3.98 (s, 3H), 3.96 (s, 3H), 3.89 (s, 3H), 3.43 (s, 6H) ppm.

¹³C NMR (101 MHz, CD₃CN): δ=156.8, 152.6, 146.4, 133.7, 122.1, 113.3, 71.2, 69.2, 62.4, 61.9, 61.7, 61.6, 57.7, 53.5, 50.0, 18.7 ppm.

### Example 2

### Stability Test

The newly designed isoindolinium salts (see, FIG. 1C) were tested for their chemical stability in dry alkaline conditions, according to the procedure described hereinabove, and the obtained data is shown in FIG. 2.

As can be seen, the chemical stability can be improved by tuning the steric effects in the aromatic ring. Compounds 12, 13 and 15 showed improved stability compared to Compound 11. Compounds 14 and 16 showed both improved life-time and better stability in dry alkaline conditions compared to BTMA.

These data support the effect of the substituents on the life-time and stability of the isoindolinium compounds, pointing towards compounds that feature one or both of the ortho positions (designated in FIG. 1B as attributing steric and electronic effect) one or two alkoxy and/or bulky halide (e.g., bromo) substituents, preferably along with one or two alkoxy and/or bulky halide (e.g., bromo) substituent(s) at one or both meta positions (designated in FIG. 1B as attributing steric effect) as exhibiting desired performance.

### Example 3

### Isoindolinium-Containing Ion Exchange Membranes

Isoindolinium compounds that exhibit a promising performance are associated with polymeric matrices, preferably incorporated into commonly used polymeric backbones such as, but not limited to, poly(phenylene oxide) (PPO); polystyrene (PS), polyethylene (PE) and poly(ethylene-co-tetrafluroethylene) (ETFE).

Membranes having a thickness of about 50 micrometers can be readily casted using PPO and PS. Membranes made of PE and ETFE can be prepared using halogen functionalized polymers via a simple S_{N}2 reaction in a polar solvent that swells but does not dissolve the membranes, and typically have a thickness of 10-50 micrometers.

Exemplary AEMs were prepared and characterized by attaching the newly designed isoindolinium compounds to a selected polymer.

An exemplary AEM was prepared using functionalized high-density PE (HDPE) and the isoindolinium Compound 17 (QA7) as follows:

### Preparation of (Trimethoxy Isoindolinium)-HDPE (TMISO-HDPE) AEM:

NaH (60% in oil, 300 mg) was slowly added to ethylene glycol (10 mL). Then, an HDPE membrane having vinylbenzyl chloride (VBC) grafted thereon was added, the flask was sealed and the reaction mixture was stirred at room temperature for 3 days. The glycol-functionalized HDPE was washed with water and ethanol, and dried under reduced pressure. The membrane was then added to a solution of Compound 17 (QA7; 300 mg) in dimethylformamide (DMF; 10 mL). The mixture was heated to 60° C. and remained at that temperature for 3 days. The membrane was then removed, washed thoroughly with ethanol and dried under vacuum. IEC measurement by AgNO₃ titration indicated an IEC of 1.6 meq/g.

### Example 4

### Performance

Membranes were further evaluated by AEMFCs tests. Polarization curves were done at different temperatures to characterize performance (power and limiting current density) and operational stability as a function of temperature. Voltage, ASR and power density were measured vs. current density at cell temperature of 60° C. with optimized dew points and 100 kPa back-pressurization on both anode and cathode; 50 and 40° C. with optimized dew points and 0 kPa back-pressurization.

Long term stability tests were performed at 60° C. under a constant current density of 300 mA/cm², with 100 kPa back-pressurization on both anode and cathode.

The newly designed anion exchange membranes were casted into AEMFC (as described hereinabove) and the ion conductivity, power density and stability were tested as described hereinabove, and further compared to a commercially available AEM FAA-3-30 [Fumasep® FAA-3-30 (26-34 micrometer thick) was obtained from Fuma Tech].

The obtained data is presented in FIGS. 3, 4A-B and 5A-B.

### Ion Conductivity

Ion conductivity of a fuel cell based on an exemplary AEM, TMISO-HDPE, over time was measured at 70° C. and 90% relative humidity as described hereinabove. The obtained data is shown in FIG. 3 and indicates that the exemplary AEM has increased conductivity over time, with a threshold of about 60 mS/cm.

In comparison, the commercially available AEM FAA-3-30 has a chloride ion conductivity of less than 10 mS/cm in similar conditions [see, Shin et al. RSC Adv., 2019, 9, 21106-21115, FIG. 8 therein].

### Voltage, ASR and Power Density

The assembly of the TMISO-HDPE based fuel cell was further characterized by studying the change of voltage, area-specific resistance, and power density as a function of current density at different temperatures (40, 50 and 60° C.). The obtained data is presented in FIG. 4A and indicate that hydroxide conductivity increases with the temperature, as does the AEMFC performance, reaching the best performance at 60° C., with a peak power density of 497 mW/cm², measured at 0.5 V, and a limiting current density of 1210 mA/cm². For comparison, an AEMFC made with commercial FAA-3-30 AEM and tested at 60° C. under the same conditions delivered a lower peak power density of 306 mW/cm² and arrived at a limiting current density of 645 mA/cm².

The average area specific resistance (ASR) through the TMISO-HDPE AEMFC decreased slightly from 0.20 measured at 40° C. to 0.16 Ωcm² at 60° C., indicating improved OH⁻conductivity. By comparison, the FAA-3-30 AEMFC showed an improved average ASR of 0.1 Ωcm² at 60° C., partly due to the reportedly higher true OH⁻conductivity at that temperature.

The performance of TMISO-HDPE at 60° C. was compared with that of the commercially available FAA-3-30, as shown in FIG. 4B, demonstrating the higher power density and reduced voltage loss of the newly designed membrane compared to FAA-3-30.

### Stability

Long term stability tests in an operating fuel cell were performed with an exemplary TMISO-HDPE in comparison with FAA-3-30. The obtained data are presented in FIG. 5A and demonstrate that the exemplary tested AEM exhibits prolonged repetitive fluctuations in voltage, which are indicative of its stability, whereby the commercially available FAA-3-30 membrane undergoes rapid degradation in the test conditions.

The AEMFCs were additionally subjected to long-term durability testing at 60° C., under a constant current density of 300 mA/cm². The TMISO-HDPE AEMFC resulted in a voltage degradation rate of 4 mV/hour after 56 hours, while the voltage of the−AEMFC dropped to 0 after 8 hours.

### Water Uptake

The obtained data are presented in FIG. 5B and demonstrate WU of the exemplary tested TMISO-HDPE reached 28% and 52% at 40 ° C. in 90% and 95% RH, respectively.

### EXAMPLE 5

### Polyrotaxanes of Polymers Featuring Quaternary Ammonium Moieties Within the Polymeric Backbone

### Synthesis

Following the polyrotaxane synthesis described by Grubbs et al. [Momčilović et al. J. Am. Chem. Soc. 2011, 133 (47), 19087-19089], four polyrotaxanes with crown ethers with increasing size as shown in FIGS. 6A, were prepared. A non-threaded control polymer 8a, lacking a crown ether, was also prepared (see, FIG. 6B).

The synthetic scheme is presented in FIG. 6B. Briefly, an ammonium diene (2a) was prepared by reaction of tert-butyl carbamate (1) with 6-bromo-l-hexene, followed by addition of TFA for deprotection and protonation. Polymerization of 2a by acyclic diene metathesis polymerization (ADMET) in the presence of 1,3-dimethoxy-5-(penetenyloxy)benzene (4) provided the exemplary QA salt polymer 5, featuring end-capping moieties at both termini. Polymer 5 was then neutralized and quaternized with methyl iodide (Mel) to provide the final polymers 8a.

In order to make the polyrotaxanes, this polymerization was repeated but 2a was pre-stirred with different crown ethers before the addition of a Hoveyda-Grubbs catalyst (3). The obtained polymers were precipitated and the ratio of crown-ether to QA was quantified by NMR (not shown). When dibenzo[24]-crown-8 (DB24C8) was used, a 1:1 ratio between 2a and the crown ether provided polymer 2d with the same 1:1 ratio. However, with other exemplary crown-ethers (DB18C6, DB21C7 and DB30C10), the crown-ether had to be added in excess in order to obtain polyrotaxane 2b, 2c and 2e (respectively) with 1:1 QA-crown ether ratio. After confirmation that all the polymers had the right amount of crown-ether, they were neutralized and quaternized with methyl iodide (MeI) to provide the final polymers 8b-8e.

Significant changes in ¹H-NMR spectra (not shown) were observed through these steps. The methylene peaks of the DB24C8 showed a slight deshielding effect upon threading with the polymer, possibly due to electrostatic interactions with the positively charged ammonium groups. In addition, the methylene peaks became broad, as typically seen in polymer peaks. Upon neutralization, all the peaks of DB24C8, including those of the aromatic moieties, showed an upfield shift, neutralizing the original shift and even surpassing the chemical shift of the free crown-ether. Finally, upon quaternization, they shifted even further upfield.

### Alkaline Stability

The alkaline stability of the polyrotaxanes and the control polymer was assessed using a protocol previously described in Dekel et al. Chem. Mater. 2017, 29 (10), 4425-4431, in which hydroxide water microsolvation is controlled.

The functionalized polymers (0.035 mmols QA) were dissolved in dry DMSO-d₆, and injected into an NMR tube having crown-ether KOH complex (4 water molecules per hydroxide) and mesitylene as internal standard. Injections were performed just before starting the NMR kinetic measurements. ¹H-NMR spectra were measured every few minutes. The decomposition process was monitored by measuring the change of the N-CH₃ peaks of the QA polymer, since it showed a clear and significant change when the amine is quaternary or tertiary.

The obtained data is shown in FIGS. 7A and 7B. Since the hydroxide is used in large excess (about 15 equivalents) compared to the QA functional groups, pseudo first-order kinetics is assumed, and a good linear fit is obtained for ln[QA] against reaction time, providing the first-order rate constant (k) for each polymer as slope (see, FIG. 7B).

The half-lives were subsequently calculated and the results are summarized in Table 1 below, showing pseudo first-order rate constants and calculated half-lives from the reaction of the polymers and hydroxide in hydration number of 4 (λ=4).

TABLE 1

| Polymer | Rate constant k [h⁻¹] | Calculated half-life t_{1/2} [h] |
|---|---|---|
| 8a | 0.0087 | 79.7 |
| 8b | 0.0058 | 119.5 |
| 8c | 0.0045 | 154 |
| 8d | 0.0026 | 266.6 |
| 8e | 0.0022 | 315 |

As clearly seen in FIGS. 7A-B and Table 1, while 8a has the same QA group as the polyrotaxanes, it presents exhibits faster kinetics, showing a QA half-life of nearly 80 hours (h). The polyrotaxanes all have a 1:1 ratio of QA to crown ether and present a trend: with the increase in crown ether size, the half-life increases as well. Polyrotaxane 8b exhibited the lowest stability among the rotaxanes, with a half-life of 119.5 hours. 8c presented a half-life of 154 hours. 8d shows 266 hours and the rotaxane with the largest CE, 8e, showed a half-life of 315 hours, about 4 times larger than the 8a. These results show that the addition of crown ethers around the positive charge can significantly enhance the kinetic stability of the QAs in the AEM.

As shown in FIG. 8, there is a correlation between stability and cavity size. The half-lives were plotted against X-ray data of the tested crown ethers, adapted from literature data [Rogers et al. J. Chem. Soc. Chem. Commun. 1987, 8, 604-606; Wiedemann, D. and Kohl, J., Acta Crystallogr. Sect. C Struct. Chem. 2017, 73 (9), 654-659; Hanson et al. J. Chem. Soc. Perkin Trans. 2 1976, No. 8, 972-976; Bush, M. A. and Truter, M. R., J. Chem. Soc. Perkin Trans. 2 1972, 3, 345-350]. For every crown ether, the largest distance between two oxygen atoms was measured. Using this correlation, one could, in principle, design additional stable polyrotaxanes by increasing the cavity size of the crown ether.

Without being bound by any particular theory, it is assumed that the increase of half-life is a result of the steric effects which block hydroxide access to the electrophilic groups around the nitrogen. Similarly to the higher ion conductivity previously reported for polyrotaxanes as related to a larger charge separation [Ge et al., 2016, supra; Yang et al., 2020, supra], a bigger barrier is present in order to prevent the hydroxide from reacting with the QA or any other positively charged groups. In addition, the crown-ethers are assumed to electrostatically repel the OH⁻due to their partial negative charge from the oxygen atoms.

### Mechanism

The mechanism of the decomposition of polyQAs in the presence of hydroxide ions, while being threaded in the cyclic ether in the polyrotaxane, was studied. It is noted that the polyQAs feature end-capping moieties so as to allow threading.

FIG. 9 presents two optional decomposition mechanisms, A and B. In mechanism A, the hydroxide may attack the QA by S_{N}2 in the methyl group. In this case, the charge is neutralized but the rotaxane remains threaded. In mechanism B, the hydroxide attacks the electrophilic carbon in the polymeric backbone or a β-hydrogen adjacent to the ammonium, which result not only in elimination and charge neutralization, but also produce a pseudo-rotaxane from which the crown-ethers can slowly unthread.

NMR studies were conducted in this regard using a model compound in which monomer 2a (see, FIG. 6B) was methylated. This molecule was reacted with base and the NMR analyses provided confirmation of Mechanism B (data not shown). The methyl hydrogens underwent the expected upfield shift, from 3.2 to 2.1 ppm, but its integration relative to the N-CH₂ peak indicated that the resulting degradation molecule retains the 2 methyl groups.

The data presented herein demonstrate the synthesis and the alkaline stability of four different exemplary polyrotaxanes and one control polymer containing QAs. The polyrotaxanes were synthesized using different dibenzo crown-ethers, with increasing cavity sizes. The results indicate that the alkaline stability of QAs is by threading them into crown ethers and that increasing the size of the crown ether leads to increased alkaline stability.

Without being bound by any particular theory, it is suggested that the increase in the alkaline stability is a result of the steric protection provided by the crown ethers which inhibits hydroxide attack, as well as the electrostatic repulsion between the crown ethers and the hydroxide. A correlation between the maximum O—O distance in the crown ether cavity and the half-life of the QAs is seen, indicating an effect of the crown-ether size on the alkaline stability.

This methodology can be used with any QAs or any other positively-charged ion conductors, for providing, for example, a practical AEM for AEMFC.

### Example 6

### Polyrotaxanes of Polymers Featuring Quaternary Ammonium Moieties Within the Pendant Groups

### Monomer Synthesis

For preparing polyrotaxanes in the polymer features quaternary ammonium groups as pendant groups, a newly designed monomer was prepared, as depicted in the following scheme. The syntheses of 36 and 37 is based on a published procedure [see, Cleveland et al. Catalysis Science& Technology, 2021, 11 (4), 1311-1322]:

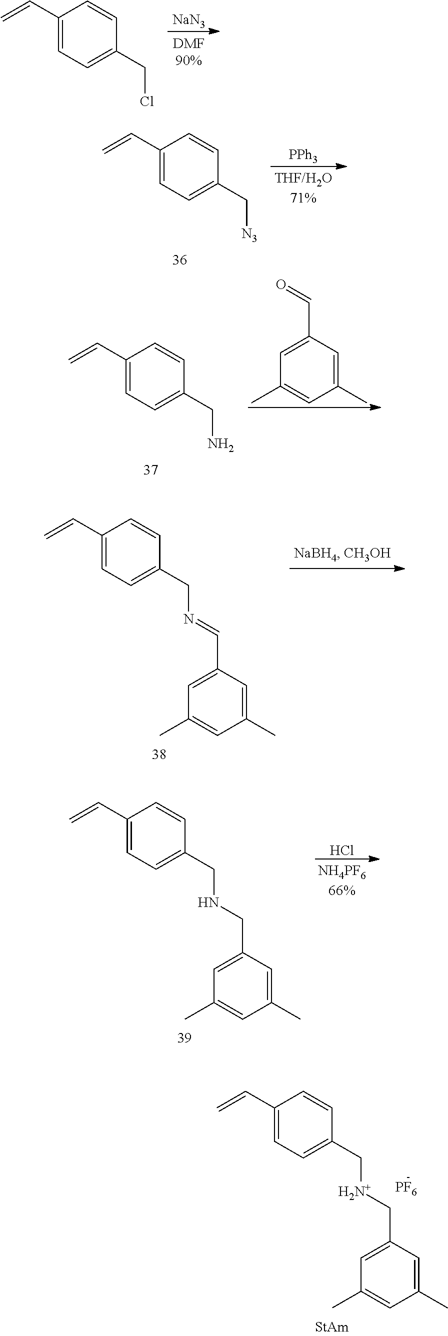

Synthesis of 36 (based on Cleveland et al. 2021, supra): 3.09 grams (0.02 mol) of 4-cholomethyl styrene and 2.86 grams (0.05 mol) of sodium azide were added into a flask (100 mL), under N₂ environment. 15 mL of anhydrous DMF were then added via a syringe, and the obtained mixture was reacted at room temperature over 5 hours, was thereafter cooled to 0° C. and subsequently poured into ice water (30 mL). Extractions of the aqueous phase were performed with diethyl ether (2×50 mL), the combined organic phase was further washed with water (80 mL) and brine (80 mL), dried over anhydrous Na₂SO₄ and concentrated under vacuum. The products were further purified by column (hexane:dichloromethane, 20:1 v/v) to afford product 36 as a colorless liquid. Yield 2.9 grams, 90%.

¹H NMR (400 MHz, D₂O): δ=7.47 (d, 2H), 7.34 (d, 2H), 6.74 (m, 1H), 5.80 (d, 1H), 5.32 (d, 1H), 4.37 (s, 2H) ppm.

Synthesis of 37 (based on Cleveland et al. 2021, supra): To a solution of Compound 36 (2.56 grams, 0.016 mmol) in anhydrous tetrahydrofuran (THF; 50 mL) was added triphenylphosphine (5.31 grams. 0.026 mol) by portions under N₂ environment. The mixture was reacted at room temperature over 24 hours. Water (5 mL) was thereafter added and stirring resumed for additional 12 hours, followed by concentration under vacuum. The crude was dissolved in aqueous HCl (1 Normal [N]), washed with dichloromethane (2×25 mL) and the aqueous phase was treated with aqueous NaOH (2 [N]) until reaching pH=10. The product was extracted using dichloromethane (2×50 mL), the organic phase was dried over Na₂SO₄ and concentrated under vacuum to afford product 37 as a pale yellow liquid product. Yield 1.5 grams, 71%.

¹H NMR (400 MHz, D₂O): δ=7.44 (d, 2H), 7.32 (d, 2H), 6.75 (m, 1H), 5.80 (d, 1H), 5.28 (d, 1H), 3.90 (s, 2H) ppm.

Synthesis of StAm: To a solution of Compound 37 (1.26 grams, 9.48 mmol) in anhydrous toluene (30 mL) was added 3,5-dimethylbenzyl aldehyde (1.27 grams, 9.48 mmol) under N₂ environment. The reaction was stirred at 45° C. overnight, anhydrous magnesium sulfate (2 grams) was added, and the reaction was stirred for additional 24 hours. After filtration, the solution was concentrated under vacuum to provide the raw material, which contained product 38, and was used as is in the next step without further purification.

The raw material containing Compound 38 was then dissolved in methanol (30 mL) and cooled to 0° C. using an ice bath. NaBH₄ (1.79 grams, 0.047 mol) was slowly added and the mixture stirred for 2 hours at room temperature. The reaction was thereafter quenched by slowly adding deionized water (4.0 mL). Following concentration under vacuum, the crude was dissolved in dichloromethane (50 mL) and washed with water (100 mL) and brine (3×50 mL). The organic phase was dried over Na₂SO₄ and concentrated under vacuum to afford a solid which contained product 39 and was used without further purifications.

To a solution of a solid containing Compound 39 in methanol (30 mL) was added equivalent HCl aqueous (2 Molar [M]) at room temperature. The mixture became clear. Saturated NH₄PF₆ aqueous solution (10 mL) was then added, followed by addition of water until the formation of an apparent white solid was observed, and the mixture was stirred overnight. The obtained white solid was thereafter filtered and washed by a large amount of water and further dried under vacuum to afford the product StAm as a white solid. Yield 2.5 grams, 66%.

¹H NMR (400 MHz, D₂O): δ=7.57 (s, 4H), 7.19 (s, 2H), 7.11 (s, 1H), 6.80 (m, 1H), 5.91 (d, 1H), 5.34 (d, 1H), 4.64 (s, 2H), 4.55 (s, 2H), 2.30 (s, 6H) ppm.

¹³C NMR (101 MHz, D₂O): δ=138.91, 138.66, 136.08, 130.91, 130.47, 127.78, 126.72, 114.85, 51.77, 51.50, 20,28 ppm.

HRMS (ESI) m/z 148.1107 (calculated C₁₀H₁₄N⁺, 148.1121).

Polymerization and quaternization: The obtained StAm was then polymerized in the presence of a crown ether, as depicted in the following scheme (“co” represents co-polymer):

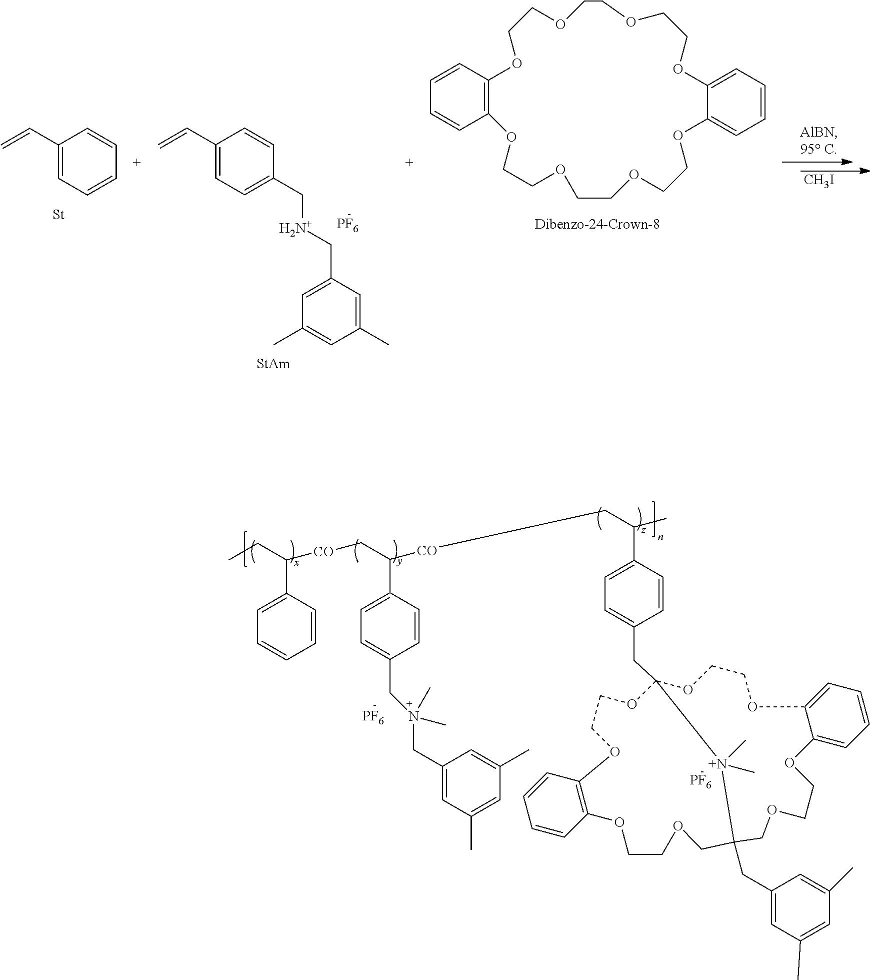

Styrene (St; feed molar ratios are specified in Table 2 below), StAm (1.16 gram, 2.91 mmol), and DB24C8 (feed molar ratios are specified in Table 2 below) were dissolved in an anisole/THF mixed solvent (5+1 mL). The mixture was sonicated for 1 minute at room temperature, providing a clear solution. Then, azobisisobutyronitrile (AIBN; 57 milligram) was added and the mixture was purged by N₂ over 20 minutes to remove the dissolved O₂. The mixture was thereafter placed in a pre-heated oil bath (95° C.), and the polymerization proceeded at this temperature for 24 hours. After cooling to room temperature, the mixture was precipitated using excess cold mixed solvent (diethyl ether/acetone, 5/1 v/v). The precipitates were collected by filtration, and the solid polymers were repeatedly washed by diethyl ether. The obtained polymers were further dried under vacuum, and the incorporation molar ratio was determined by NMR (not shown). The obtained data is presented in Table 2.

The obtained polymers (1 mol equivalent of the ammonia group) was dissolved in anhydrous DMF, and methyl iodide (20 mol equivalents) and K₂CO₃ (10 mol equivalents) were added. The mixture was reacted at room temperature over 24 hours and thereafter was directly poured into mixed cold solvent (diethyl ether/methanol/water, 10/2/1 v/v). The obtained polyrotaxanes were collected by filtration and dried under vacuum, to provide the polyrotaxanes in moderate yields.

Following the above-described procedure, polyrotaxanes obtained while using varying mol ratios of the reacting materials were prepared, as presented by Table 2 below.

TABLE 2

| | | Incorporation | Incorporation | |
|---|---|---|---|---|
| | Feed molar ratio | molar ratio | molar ratio | |
| | of [St]/[StAm]/ | pre- | following | Yield |
| Polymer | [DB24C8] | methylation | methylation | (%) |
| P1 | 3/1/0 | 3.4/1/0 | 3.4/1/0 | 62 |
| P2 | 3/1/0.17 | 1.7/1/0.16 | 1.7/1/0.16 | 38 |
| P3 | 3/1/0.34 | 2.7/1/0.45 | 2.23/1/0.54 | 57 |
| P4 | 3/1/1.5 | 3.3/1/1 | 3.3/1/1 | 37 |

Table 2 shows that the polymerizations led to a change in molar ratio between St, StAm and the crown-ether DB24C8 at all the tested polyrotaxanes P1-P4. Methylation had minor to no influence on molar ratio in P1, P2 and P4, but P3 showed a certain change in molar ratio.

NMR analyses (data not shown) showed that the methylene peaks become broad post-methylation, similar to the case of the polyrotaxanes described in Example 5 hereinabove.

These polyrotaxanes are further studied in alkaline stability tests [Dekel, 2017, supra], and in AEM and AEM-fuel cell tests as described in Examples 2-4 hereinabove.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An ion conductor comprising a polymeric matrix and a quaternary ammonium salt-containing moiety being in association with said matrix, said quaternary ammonium salt-containing moiety being represented by Formula I: 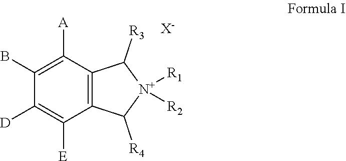
wherein:
R₁ and R₂ are each independently selected from alkyl, cycloalkyl and aryl, or represents an attachment point to the polymeric matrix;
X⁻is a counter anion;
R₃, R₄, A and E are each independently selected from hydrogen, alkyl, halo, haloalkyl, cycloalkyl, aryl, heteroaryl, hydroxy, alkoxy, aryloxy, and any other substituent, or represents an attachment point to the polymeric matrix, provided that at least one of A and E is a bulky substituent capable of imparting steric hindrance around the positively charged portion of the compound; and
B and D are each independently hydrogen, halo, alkyl, haloalkyl, cycloalkyl, aryl, heteroaryl, hydroxy, alkoxy, aryloxy, and any other substituent, or represents an attachment point to the polymeric matrix, provided that at least one of B and D is a bulky substituent that positions one or both of A and E at a configuration that imparts said steric hindrance.

2. The ion conductor of claim 1, wherein at least one of B and D is other than hydrogen or fluoro.

3. The ion conductor of claim 1, wherein at least one of B and D is chloro, bromo, or iodo.

4. The ion conductor of claim 1, wherein at least one of B and D is alkoxy.

5. The ion conductor of claim 1, wherein at least one, or each, of A and E is alkoxy.

6-7. (canceled)

8. The ion conductor of claim 1, wherein at least one or both of A and E is independently an aryl.

9. The ion conductor of claim 1, comprising a polymeric or co-polymeric backbone which comprises a plurality of backbone units, wherein at least a portion of said backbone units comprise said quaternary ammonium salt-containing moiety represented by Formula I, and wherein an attachment point of said moiety to said backbone units in via one or more of A, B, D, E, R₁, R₂, R₃ or R₄.

10. The ion conductor of claim 9, wherein said quaternary ammonium salt-containing moiety represented by Formula I is covalently attached as a pendant group to said portion of backbone units or forms a part of said polymeric backbone.

11-12. (canceled)

13. An anion conducting composition comprising an ion conductor as defined in claim 1 and a cyclic moiety associated with said ion conductor.

14. The anion conducting composition of claim 13, wherein said cyclic moiety is mechanically interlocked around at least a portion of said ion conductor.

15. (canceled)

16. The anion conducting composition of claim 13, wherein said cyclic moiety is a heterocyclic moiety which comprises at least 12 carbon atoms and at least one electronegative heteroatom.

17-19. (canceled)

20. An anion exchange membrane comprising the ion conductor claim 1.

21. An electrochemical system comprising the anion exchange membrane of claim 20.

22. (canceled)

23. An article-of-manufacturing comprising the ion conductor claim 1.

24. An anion conducting composition comprising an anion conducting polymer featuring at least one end-capping moiety at a backbone terminus thereof and/or at a pendant group terminus thereof and a cyclic moiety, said polymer being threaded within a cyclic moiety, wherein said at least one end-capping moiety has a volume larger than a volume of said cyclic moiety to thereby have said cyclic moiety mechanically interlocked around said linear polymer, wherein said cyclic moiety is a heterocyclic moiety that comprises at least 12 carbon atoms and at least one electronegative heteroatom.

25-27. (canceled)

28. The anion conducting composition of claim 24, wherein said cyclic moiety is a crown ether.

29. (canceled)

30. The anion conducting composition of claim 24, wherein said cyclic moiety is covalently attached to said polymer.

31-32. (canceled)

33. An anion exchange membrane comprising the anion conducting composition of claim 24.

34. An electrochemical system comprising the anion exchange membrane of claim 33.

35. (canceled)

36. An article-of-manufacturing comprising the anion conducting composition of claim 24.

37. (canceled)
